(19) United States
(12) Patent Application Publication
(10) Pub. No.: US 2025/0345316 A1
(43) Pub. Date: Nov. 13, 2025

(54) METHODS OF INCREASING LYMPHOCYTE TRAFFICKING TO THE COLON BY STIMULATING RETINOIC ACID RECEPTORS AND ARYL HYDROCARBON RECEPTORS

(71) Applicant: President and Fellows of Harvard College, Cambridge, MA (US)

(72) Inventors: Ziqi Chen, Cambridge, MA (US); Flora Mary Tierney, Cambridge, MA (US); Ulrich von Andrian, Chestnut Hill, MA (US)

(73) Assignee: President and Fellows of Harvard College, Cambridge, MA (US)

(21) Appl. No.: 19/201,209

(22) Filed: May 7, 2025

Related U.S. Application Data
(60) Provisional application No. 63/644,047, filed on May 8, 2024.

(51) Int. Cl. A61K 31/437; A61K 31/196; A61K 45/06; A61P 31/04

(52) U.S. Cl. CPC A61K 31/437; A61K 31/196; A61K 45/06; A61P 31/04

(57) ABSTRACT

The technology described herein is directed to modulate the levels of CD8+ T cells in the colon and/or small intestine including administering to a subject in need thereof a therapeutically effective amount of a retinoic acid receptor (RAR) agonist or inhibitor and/or an aryl hydrocarbon receptor (AHR) agonist or inhibitor, and a therapeutic agent.

### RELATED APPLICATIONS

This application claims the benefit of priority to U.S. Provisional Application No. 63/644,047, filed on May 8, 2024, the entire contents of which are incorporated herein by reference.

### TECHNICAL FIELD

Described herein are compositions and methods related to targeting and treating diseases and disorders of the gut, such as the small intestine and colon, increasing lymphocyte trafficking to the gut by with retinoic acid receptors (RAR) and aryl hydrocarbon receptors (AHR) agonists.

### BACKGROUND OF THE INVENTION

While naïve T cells circulate through the blood, the lymph and secondary lymphoid organs, antigen-experienced T cells can migrate to sites where the antigen is seen and carry out their immune functions. The trafficking of T cells to peripheral tissues, or “T cell homing”, is a multistep process dependent on specific adhesion receptors and chemokine receptors on T cell surfaceⁱ. In tissues such as skinⁱⁱ and the small intestineⁱⁱⁱ, these receptors are identified to be regulated by the environment of the secondary lymphoid organs during antigen stimulation, in a process termed “lymphocyte imprinting”. In the small intestine, imprinting is mediated by retinoic acid, a metabolite of vitamin A, through activation of retinoic acid receptor (RAR) and up-regulation of homing receptors α4β7 and CCR9^{iv,v}. While a similar tissue-specific mechanism has been proposed for the colon^{vi}, the actual tissue-specific environmental cues remain to be defined. Lines of evidence have suggested that colon homing is partly dependent on α4, β7 and the chemokine receptor GPR15^{vii,viii}. Notably, retinoic acid signaling has been reported to upregulate α4β7 expression^{ix}, while aryl hydrocarbon receptor (AHR) signaling has been reported to upregulate GPR15 expression on CD4+ T cells and increase CD4+ T cell homing to the colon^{x,xi}.

### SUMMARY OF THE INVENTION

The present invention provides methods of increasing the level of CD8+ T cells in the colon and/or small intestine, the method comprising administering to a subject in need thereof a therapeutically effective amount of a retinoic acid receptor (RAR) agonist and/or an aryl hydrocarbon receptor (AHR) agonist, thereby increasing the level CD8+ T cells in the colon and/or small intestine compared to a control.

In another aspect, the present invention is directed to methods of increasing trafficking of CD8+ T cells to the colon and/or small intestine, the method comprising administering to a subject in need thereof a therapeutically effective amount of a retinoic acid receptor (RAR) agonist and/or an aryl hydrocarbon receptor (AHR) agonist, thereby increasing trafficking of CD8+ T cells to the colon and/or small intestine compared to a control.

In some embodiments, the method further comprises administering to the subject a therapeutic agent.

In some embodiments, the level of CD8+ T cells is increased between about 5% to about 70%, about 10% to about 50%, about 20% to about 30%, about 55% to about 65%, or about 15% to about 40% in the colon and/or small intestine compared to a control. In some embodiments, the level of CD8+ T cells is increased at least about 20%, about 25%, about 35%, about 40%, about 45%, about 50%, about 55%, about 60%, or about 65% in the colon and/or small intestine compared to a control.

In some embodiments, the control comprises a level of CD8+ T cells in the colon and/or small intestine prior to the administration of RAR agonists and/or AHR agonists.

In some embodiments, the CD8+ T cells in the colon and/or small intestine express CD69+CD103+. In some embodiments, between about 20% to about 100%, about 30% to about 60%, about 40% to about 60%, about 30% to about 70%, about 60% to about 80% of the CD8+ T cells in the colon and/or small intestine express CD69+CD103+.

In some embodiments, the CD8+ T cells in the colon and/or small intestine express one or more of α4β7, GPR15, and/or CCR9. In some embodiments, between about 20% to about 100%, about 30% to about 60%, about 40% to about 60%, about 30% to about 70%, about 60% to about 80% of the CD8+ T cells in the colon and/or small intestine express α4β7 compared to a level of CD8+α4β7+ T cells in the colon and/or small intestine prior to the administration of RAR agonists and/or AHR agonists.

In some embodiments, between about 20% to about 100%, about 30% to about 60%, about 40% to about 60%, about 30% to about 70%, about 60% to about 80% of the CD8+ T cells express GPR15 compared to a level of CD8+GPR15+ T cells in the colon and/or small intestine prior to the administration of RAR agonists and/or AHR agonists.

In some embodiments, between about 20% to about 100%, about 30% to about 60%, about 40% to about 60%, about 30% to about 70%, about 60% to about 80% of the CD8+ T cells express CCR9 compared to a level of CD8+CCR9+ T cells in the colon and/or small intestine prior to the administration of RAR agonists and/or AHR agonists.

In some embodiments, further wherein the level of circulating memory T cells expressing CXCR3 and CX3CR1 are increased in the blood compared to a level of CXCR3+CX3CR1+ memory T cells in the blood prior to the administration of RAR agonists and/or AHR agonists.

In another aspect, the present invention is directed to methods of increasing the efficacy of a therapeutic agent, the method comprising administering a retinoic acid receptor (RAR) agonist and/or an aryl hydrocarbon receptor (AHR) agonist before, concomitantly, or after administering the therapeutic agent, thereby increasing the efficacy of the therapeutic agent compared to a control.

In some embodiments, the control comprises a level of efficacy of the therapeutic agent prior to the administration of RAR agonists and/or AHR agonists.

In another aspect, the present invention is directed to methods for treating a disease or disorder of the colon and/or small intestine, the method comprising administering to a subject in need thereof a therapeutically effective amount of a retinoic acid receptor (RAR) agonist and/or an aryl hydrocarbon receptor (AHR) agonist, thereby treating the disease or disorder of the colon and/or small intestine.

In some embodiments, the method, further comprises administering to the subject a therapeutic agent.

In some embodiments, the disease or disorder of the colon or small intestine is a cancer, an autoimmune disease, or an enteric pathogenic infection. In some embodiments, the cancer is colorectal cancer, adenocarcinoma, sarcoma, carcinoid tumor, gastrointestinal stromal tumor (GIST), or intestinal lymphoma. In some embodiments, the autoimmune disease is Crohn's disease, ulcerative colitis, celiac disease, autoimmune enteropathy, eosinophilic colitis, Behcet's disease, or autoimmune gastritis.

In some embodiments, the enteric pathogenic infection is Salmonella, Shigella, Clostidioides difficile, Campylobacter jejuni, Vibrio cholera, Yersinia enterocolitica, Escherichia coli, Listeria monocytogenes, Rotavirus, Poliovirus, or Norovirus. In some embodiments, the enteric pathogenic infection is Listeria monocytogenes.

In some embodiments, the therapeutic agent is a vaccine, anti-inflammatory, chemotherapeutic, probiotic and/or an antibiotic.

In some embodiments, the RAR agonist is selected from the group consisting of all-trans retinoic acid (ATRA), AM80, AM580, AC 261066, Adapalene, BMS 753, BMS 961, CD 1530, CD2314, CD437, ch 55, DC271, retinoic acid, TTNPB, etretinate, tazarotene, tamibarotene, and a combination thereof.

In some embodiments, the AHR agonist is selected from the group consisting of 10-CL-BBQ, L-kynurenine, ITE, FICZ, indirubin, VAF347, a flavonoid, a carotinoid, a glucobrassin metabolite, a tryptophan metabolite, and a combination thereof.

In some embodiments, administering to the subject the RAR agonist. In some embodiments, administering the RAR agonist results in an increase in the level of CD8+ T cells in the small intestine. In some embodiments, administering to the subject the AHR agonist. In some embodiments, administering the AHR agonist results in an increase in the level of CD8+ T cells in the colon. In some embodiments, the method comprises administering a combination of the RAR agonist and the AHR agonist to the subject.

In some embodiments, the RAR agonist and/or the AHR agonist are encapsulated in a nanoparticle comprising one or more polymers. In some embodiments, the one or more polymers comprise polylactic-co-glycolic acid (PLGA), polyethylene glycol (PEG), poly F-caprolactone (PCL), poly lactic acid (PLA), chitosan, dextran, acetylated-dextran (AcDex), or a combination thereof. In some embodiments, the one or more polymers comprise AcDex.

In some embodiments, the RAR agonist and/or the AHR agonist, or the RAR agonist and/or the AHR agonist encapsulated in a nanoparticle are formulated for subcutaneous injection.

In some embodiments, the RAR agonist and/or the AHR agonist and the therapeutic agent are administered concomitantly. In some embodiments, the RAR agonist and/or the AHR agonist and the therapeutic agent are administered sequentially. In some embodiments, the RAR agonist and/or the AHR agonist are administered after the therapeutic agent. In some embodiments, the RAR agonist and/or the AHR agonist are administered before the therapeutic agent.

In some embodiments, the RAR agonist and/or the AHR agonist are administered twice a day, once a day, once every two days, once a week, twice a week, once a month, once every two months, or once every six months. In some embodiments, the RAR agonist and/or the AHR agonist are administered for 1 week to 1 year.

In some embodiments, the RAR agonist and the AHR agonist are administered separately. In some embodiments, the RAR agonist is administered prior to the AHR agonist. In some embodiments, the AHR agonist is administered for more doses than the RAR agonist.

In another aspect, the present invention is directed to methods of decreasing the level of CD8+ T cells in the colon and/or small intestine, the method comprising administering to a subject in need thereof a therapeutically effective amount of a retinoic acid receptor (RAR) inhibitor and/or an aryl hydrocarbon receptor (AHR) inhibitor, thereby decreasing the level CD8+ T cells in the colon and/or small intestine compared to a control.

In another aspect, the present invention is directed to methods of decreasing trafficking of CD8+ T cells to the colon and/or small intestine, the method comprising administering to a subject in need thereof a therapeutically effective amount of a retinoic acid receptor (RAR) inhibitor and/or an aryl hydrocarbon receptor (AHR) inhibitor, thereby decreasing trafficking of CD8+ T cells to the colon and/or small intestine compared to a control.

In another aspect, the present invention is directed to methods for treating an inflammatory disease or disorder of the colon and/or small intestine, the method comprising administering to a subject in need thereof a therapeutically effective amount of a retinoic acid receptor (RAR) inhibitor and/or an aryl hydrocarbon receptor (AHR) inhibitor, thereby treating the inflammatory disease or disorder of the colon and/or small intestine.

In some embodiments, the method further comprises administering to the subject a therapeutic agent. In some embodiments, the control comprises a level of CD8+ T cells in the colon and/or small intestine prior to the administration of the RAR inhibitor and/or the AHR inhibitor. In some embodiments, the inflammatory disease or disorder of the colon or small intestine an autoimmune disease.

In some embodiments, the RAR inhibitor is selected from the group consisting of 4-[(1E)-2-[5,6-Dihydro-5,5-dimethyl-8-(phenylethynyl)-2-naphthalenyl]ethenyl]-benzoic acid (BMS 493), 4-[2-(5,6,7,8-Tetrahydro-5,5,8,8-tetramethyl-2-naphthalenyl)ethynyl)-benzoic acid (EC 23), AGN 193109-d7,4-[2-[5,6-Dihydro-5,5-dimethyl-8-(4-methylphenyl)-2-naphthalenyl]ethynyl]benzoic Acid Sodium Salt (AGN 193109 Sodium Salt), 4-[[[5,6-Dihydro-5,5-dimethyl-8-(3-quinolinyl)-2-naphthalenyl]carbonyl]amino]benzoic acid (BMS 1695614), 4-[6-[(2-Methoxyethoxy)methoxy]-7-tricyclo[3.3.1.13,7]dec-1-yl-2-naphthalenyl)benzoic acid (CD2665), 4-[5-[8-(1-Methylethyl)-4-phenyl-2-quinolinyl]-1H-pyrrolo-2-benzoic acid (ER50891), 4-(7,8,9,10-Tetrahydro-5,7,7,10,10-pentamethyl-5H-benzo[e]naphtho[2,3-b][1,4]diazepin-13-yl)benzoic acid (LE135), 4-[5-[3,5-Bis(1,1-dimethylethyl)phenyl]-1-[4-[(4-methyl-1-piperazinyl)carbonyl]phenyl]-1H-pyrazol-3-yl]benzoic acid (LY2955303), 6-[2-(5,6,7,8-Tetrahydro-5,5,8,8-tetramethyl-2-naphthalenyl)-1,3-dithiolan-2-yl]-2-naphthalenecarboxylic acid (MM11253), UVI3003, and a combination thereof.

In some embodiments, the AHR inhibitor is selected from the group consisting of (S)-6-(4-chlorophenyl)-N-(1-hydroxypropan-2-yl)-2-(1-methyl-1H-pyrazol-4-yl)-3-oxo-2,3-dihydropyridazine-4-carboxamide (BAY 2416964), (R)-N-(2-(5-fluoropyridin-3-yl)-8-isopropylpyrazolo[1,5-a][1,3,5]triazin-4-yl)-2,3,4,9-tetrahydro-1H-carbazol-3-amine (IK-175), N-(2-(1H-indol-3-yl)ethyl)-5-(5-fluoropyridin-3-yl)-3-methylpyrazolo[1,5-a]pyrimidin-7-amine (KYN-101), (1S,2S)-2-(3,4-dichlorobenzoyl)cyclopropane-1-carboxylic acid (UPF-648), brevifolincarboxylic acid, 26-Deoxyactein, hCYP1B1-IN-2, AHR-IN-1,1,2,3,4,7,8,9-Heptachlorodibenzofuran (1,2,3,4,7,8,9-HpCDF), 1,2,3,4,7,8-Hexachlorodibenzofuran (1,2,3,4,7,8-HxCDF), and a combination thereof.

### BRIEF DESCRIPTION OF THE DRAWINGS

FIGS. 1A-1C depicts a schematic of parenteral vaccination to target CD8+ lymphocytes to the small and large intestine for total intestinal tract protection. FIG. 1A shows no gut imprinting. FIG. 1B shows treatment with ATRA vaccine and Am80 resulting in production of mucosal antibodies and targeting CD8+ T lymphocytes to the small intestine. FIG. 1C shows treatment with ATRA vaccine, Am80 and an AHR ligand resulting in production of mucosal antibodies and targeting CD8+ T lymphocytes to the small and large intestine.

FIG. 2 depicts a schematic of an experimental approach. Antigen-specific CD8+ T cells (OT-1 cells, recognizing a peptide within the chicken ovalbumin protein) were adoptively transferred (1×10⁵ cells) to B6 recipient mice on day −1, and B6 recipients were vaccinated subcutaneously (s.c.) on day 0 with 100 μg chicken ovalbumin (OVA) and an adjuvant CpG-1668 (0.5 nmol). On day 0.1 and 2, mice were injected subcutaneously with solvent control (DMSO, 10% v/v in PEG-400), retinoic acid receptor agonist Am80 (500 nmol in PEG-400), AHR agonist 10-CL-BBQ (164 nmol in PEG-400), or Am80+10-CL-BBQ (same dose). Tissues were harvest on day 7 and analyzed by flow cytometry.

FIGS. 3A-3G While Am80 or 10-CL-BBQ alone induced some increase of OT-1 effector cells in the colon and cecum, combination of Am80+10-CL-BBQ were the most effective at increasing effector T cells responses in the colon and cecum. Either Am80 alone or Am80+10-CL-BBQ increased OT-1 cells in the small intestine, whereas 10-CL-BBQ alone had no effect. The effects observed in the intestinal tissues were not due to overall higher immune response, as the number and percentage of OT-1 cells in the spleen were similar across all treatment conditions. Percentage of OT-1 cells out of total CD8a⁺ T cells in the spleen (FIG. 3A), colon (FIG. 3B), cecum (FIG. 3C) and small intestine (FIG. 3D); Total number of OT-1 cells recovered from the spleen (FIG. 3E), colon (FIG. 3F) and cecum (FIG. 3G). Brown-Forsythe and Welch's ANOVA with Dunnett's T3 multiple comparisons test. *p<0.05, **p<0.01, ***p<0.001, and ****p<0.0001. ns, not significant.

FIG. 3H AM80 and 10-Cl-BBQ synergistically targets OT-I T cells to the colon. Wildtype C57B6 recipient mice were adoptively transferred with OT-I cells from CD45.1+ congenic donors, and 6 hours later all recipients were immunized subcutaneously (scruff) with OVA and CpG (day 0). On day 0, 1 and 2, mice were injected subcutaneously (scruff) with Am80, 10-CL-BBQ, AM80+10-CL-BBQ or ethanol (negative control). AM80 is a retinoic acid receptor agonist and was injected once daily, stocked in ethanol, and formulated in PEG-400 to a final dose of 175 ug/100 ul. 10-CL-BBQ is an AHR agonist and was injected twice daily, stocked in ethanol, and formulated in PEG-400 to a final dose of 50 ug/100 ul. Spleen and colon were collected at 6 days post vaccination; data is from 2 independent experiments. Left: percentage of OT-I cells among CD8a+ cells. Right: Total OT-I T cell numbers recovered. Combination of AM80 and 10-CL-BBQ significantly increased the number of transferred OT-I cells in the colon (unpaired t-test with Welch's correction, comparing AM80+10-CL-BBQ to each other group).

FIG. 3I depicts quantification of flow cytometry analysis of the spleen, small intestine IEL, and small intestine LPL on day 7 after treatment with an RAR agonist. FIG. 3J depicts quantification of cells/0.05 mm³ on day 7 after treatment with an RAR agonist.

FIGS. 4A-4D Using the same vaccination regimen described in FIG. 2, blood was analyzed at timepoints between day 4-6 by flow cytometry. FIG. 4A shows a representative flow cytometry plots showing expression of α4β7 and GPR15 on OT-1 cells in the blood. Plots are pre-gated on OT-1 cells. As shown, 10-CL-BBQ alone induced the most robust GPR15 expression, while Am80+10-CL-BBQ induced the most robust α4β7+GPR15+ cells. FIG. 4B depicts a schematic of experiment. FIG. 4C shows the percentage of GPR15+ cells out of total OT-1 cells. FIG. 4D shows the percentage of α4β7+GPR15+ cells out of total OT-1 cells. Brown-Forsythe and Welch's ANOVA with Dunnett's T3 multiple comparisons test. *p<0.05, **p<0.01, ***p<0.001, and ****p<0.0001. ns, not significant.

FIG. 5 Using the same vaccination regimen described in FIG. 2, blood was analyzed at day 5 by flow cytometry. Representative flow cytometry plots showing expression of GPR15 on adoptively transferred OT-1 cells (GFP+) that's in the effector phase, and CD8+ T cells from the recipient animal (GFP−) that's mostly naïve. Plots are pre-gated on CD8a+ cells. As shown, 10-CL-BBQ mostly up-regulated GPR15 on OT-1 cells but did not affect GPR15 expression on CD8a+ cells in the blood of the recipient.

FIG. 6 Using the same vaccination regimen described in FIG. 2, blood was analyzed at day 5 by flow cytometry. Representative flow cytometry plots of the AM80+10CLBBQ group showing expression of CCR9 and GPR15 on adoptively transferred OT-1 cells (GFP+), and in OT-1 cells that are α4β7+. Expression of CCR9 and GPR15 does not appear to be interacting with each other.

FIG. 7 Using the same adoptive transfer, immunization and treatment regimen, draining lymph nodes (brachial LNs) were harvested at different days to analyze the expression of gut homing molecules. Representative flow cytometry plots (pre-gated on OT-1 cells) from brachial lymph nodes of Am80+10-CL-BBQ treated mice. While CCR9 was induced within 24 hrs, the upregulation of GPR15 and generation of α4β7+GPR15+ cells started at day 4 (96 hours) in the LNs. Upregulation of GPR15 happened at the peak of α4β7 expression, and concurrently with the decline of CCR9 expression.

FIGS. 8A-8B L-kynurenine (L-KYN), an endogenous AHR ligand, was tested for the ability to up-regulate GPR15 and promote large intestine trafficking in the parenteral vaccination model. L-KYN, at the experimental dose (500 nmol in PEG-400) and regimen, did not increase expression of GPR15 on effector CD8+ T cells. FIG. 8A shows a schematic of experiment. FIG. 8B shows a representative flow cytometry plot of day 4 blood, pre-gated on OT-1 cells. While 10-CL-BBQ significantly up-regulated GPR15 on OT-1 cells, L-KYN did not have any effect.

FIG. 9 AHR ligand L-kynurenine (L-KYN), at the dosing regimen, did not increase colon trafficking alone or in combination with Am80. FIG. 9 shows the percentage of OT-1 cells out of total CD8+ T cells, in the spleen and colon following vaccination and respective treatments.

FIGS. 10A-10B Testing AhR Agonist V, VAF347, a synthetic AHR agonist, for the ability to promote large intestine trafficking in the parenteral vaccination model. VAF347 at an experimental dose (250 nmol in PEG-400) and regimen, increased colon trafficking but at a lower efficacy than 10-CL-BBQ. Additionally, injection of CH-223191, an AHR antagonist (250 nmol in PEG-400), antagonized the effects of 10-CL-BBQ on colon trafficking. FIG. 10A shows a schematic of experiment. FIG. 10B shows the percentage of OT-1 cells out of total CD8⁺ T cells, in the spleen and colon following vaccination and respective treatments.

FIGS. 11A-11D show in vitro culture method to generate GPR15⁺CD8+ T cells. Various treatment conditions were tested for the efficiency of generating GPR15⁺CD8⁺ T cells. OT-1 splenocytes were pulsed for 1 hour with 1 μg OVA peptide (257-264) and cultured overnight in complete RPMI media. The next day, CD8⁺ T cells were isolated using mouse CD8a⁺ T Cell Isolation Kit (Miltenyi) and cultured with various treatment conditions. In summary, combination of mouse IL-2 (5 ng/ml), TGF-β1 (5 ng/ml) and 10 nM 10-CL-BBQ yielded the most robust results. ***Particularly with the in vitro culture, there were some fluctuations between experiments on how much GPR15 was expressed, but overall conclusion remained the same. FIG. 11A shows an example flow cytometry plots of GPR15 expression in various treatment conditions. 4 days post activation. Plots were pre-gated on CD8a⁺ cells. FIG. 11B shows the percentage of GPR15⁺ cells out of total CD8+ T cells, 4-5 days post activation. FIG. 11C shows the effect of different doses of 10-CL-BBQ, with IL-2, with or without TGF-β1, 4-5 days post activation. FIG. 11D shows an experiment showing percentage of GPR15⁺ cells out of total CD8⁺ T cells over time. GPR15⁺ T cells peaked around 4-5 days post activation. Cells were treated with IL-2+10-CL-BBQ+TGF-β1.

FIG. 12 depicts a schematic of homing and competitive homing assays using OT-1 cells. Briefly, two GFP+OT-1 transgenic mice with or without expression of the congenic marker CD45.1 were used per experiment (mouse A and mouse B), and OT-1 cells were isolated from their spleens. OT-1 cells from different mice ([A] and [B]) were subjected to different treatments (A and B, including culture, genetic manipulation, in vivo activation and re-harvesting, etc). The cells were then mixed at an around 1:1 ratio and injected into a B6 recipient (GFP−). At timepoints (determined for each experiment), with or without additional in vivo treatments (determined by each experiment), tissues were analyzed by flow cytometry and the ratio of [A] and [B] in each tissue was obtained. Homing index and normalized homing index (see equation) were calculated. A high homing index [A/B](>1) means [A] is over-represented in the analyzed tissue compared to [B], suggesting that the treatments that [A] underwent made them better at trafficking to and staying in the analyzed tissue.

FIGS. 13A-13B Cells were either cultured with IL-2 alone, or with the “optimized” method for GPR15 expression (IL-2+10-CL-BBQ+TGF-β1). The two culture conditions were distinguished by expression of congenic markers 45.1 and mixed cells (5×10⁶ cells from each treatment) were transferred into naïve B6 recipients. While the previously described method generated GPR15+ cells robustly compared to IL-2 alone, neither condition generated cells capable of trafficking to the gut, likely due to low levels of α4β7 expression. FIG. 13A shows expression of α4β7 and GPR15 in input cells (cells treated with IL-2 alone were 45.1+, and cells treated with the “optimized” method was 45.1−). FIG. 13B shows a representative plots of the spleen, small intestine (IEL and LPL), colon and cecum. Transferred cells were gated on GFP and CD45.1. While both populations were detected in the spleen, none of the transferred cells could be detected across all intestinal compartments.

FIG. 14 10 nM all-trans retinoic acid (ATRA) was added to the cell culture at different time points, and characterized the expression of α4β7, GPR15 and CCR9 at day 4 post activation. While adding ATRA concurrently with IL-2+ TGF-β1+10-CL-BBQ completely abrogated GPR15 expression and skewed cells towards a small intestine trafficking phenotype (α4β7^{high}CCR9+), adding ATRA 24-48 hours after IL-2+ TGF-β1+10-CL-BBQ enabled the generation of an α4β7^{high}GPR15+ population.

FIG. 15 α4β7^{high} GPR15+ cells were generated using the method described in FIG. 14 and transferred these cells (1×10⁷ cells) into a naïve B6 recipient. In the spleen, small intestine (IEL and LPL), colon and cecum, the transferred cells can be gated (plots pre-gated on OT-1 cells). Furthermore, OT-1 cells recovered from the colon and cecum preferentially showed increased GPR15 expression. This lends additional support to GPR15 playing a role in CD8+ T cell trafficking to the large intestine.

FIG. 16 depicts an example of flow cytometry plots from a competitive homing experiment, comparing Am80+10-CL-BBQ versus DMSO. As shown, compared to cell ratio in the spleen and blood, OT-1 cells from the Am80+10-CL-BBQ treated animals were significantly over-represented in all intestinal tissues.

FIG. 17 depicts summary plots of in vivo competitive homing (described previously). OT-1 cells from combined treatment group (Am80+10-CL-BBQ) showed preferential trafficking to the colon and cecum when compared to any other treatment condition (DMSO, Am80 alone, 10-CL-BBQ alone). Combined treatment group also showed preferential trafficking to the small intestine (IEL and LPL) when compared to DMSO and 10-CL-BBQ groups, but not compared to Am80 group. Normalized to input ratio. Kruskal-Wallis test and Dunn's multiple comparisons test. *p<0.05, **p<0.01, ***p<0.001, and ****p<0.0001. ns, not significant.

FIGS. 18A-18D Loss of GPR15 gene by CRISPR knockout abrogated GPR15 expression but did not affect expression of α4β7 and CCR9. Naïve GFP+OT-1 cells were nucleofected with Cas9 protein, complexed with either control (CD19) gRNA, or GPR15 gRNA. Transfected cells were distinguished based on expression of congenic marker CD45.1 and co-transferred into recipient mice. Recipient mice were immunized with OVA+CpG, and injected with DMSO, Am80, 10-CL-BBQ or Am80+10-CL-BBQ. FIG. 18A depicts representative flow cytometry plots, gated on OT-1 cells. At day 5 in the blood, OT-1 cells nucleofected with GPR15 gRNA (CD45.1+) almost fully abrogated GPR15 expression, compared to OT-1 cells nucleofected with control gRNA (CD45.1−). Quantification of GPR15+(FIG. 18B), α4β7+(FIG. 18C) and CCR9+ cells (FIG. 18D), percentage out of OT-1 cells in respective groups. Ctrl cells: CD45.1− and GPR15 knockout cells: CD45.1+.

FIG. 19 depicts a bar graph showing decreased trafficking of OT-1 cells to spleen, bLN, SI IEL, SI LPL, cecum, and colon in mice 7 days after immunization. Ratio of [Ctrl]:[GPR15 knockout] and normalized to spleen.

FIG. 20 depicts bar graphs showing level of trafficking of OT-1 cells to bLN, SI IEL, SI LPL, cecum, and colon in mice 7 days after immunization and treatment with either DMSO, Am80, 10-CL-BBq, or Am80+CL-10-BBQ. Consistent with the level of GPR15 expression, at day 7, GPR15 KO cells were under-represented in the colon and cecum in all groups. Ratio of [Ctrl]:[GPR15 knockout] and normalized to spleen. Kruskal-Wallis test and Dunn's multiple comparisons test. *p<0.05, **p<0.01, ***p<0.001, and ****p<0.0001. ns, not significant.

FIGS. 21A-21E Loss of AHR abrogated ligand-depended upregulation of GPR15. Naïve GFP+OT-1 cells were nucleofected with Cas9 protein, complexed with either control (CD19) gRNA, or AHR gRNA. Transfected cells were distinguished based on expression of congenic marker CD45.1 and co-transferred into recipient mice. Recipient mice were immunized with OVA+CpG, and injected with DMSO, Am80, 10-CL-BBQ or Am80+10-CL-BBQ (same dose and regimen as previously described). FIG. 21A shows a representative flow cytometry plots, gated on OT-1 cells. At day 5 in the blood, OT-1 cells nucleofected with AHR gRNA (CD45.1+) showed baseline expression of GPR15, but not 10-CL-BBQ dependent upregulation of GPR15, compared to OT-1 cells nucleofected with control gRNA (CD45.1−). Quantification is shown of GPR15+(FIG. 21B), α4β7+(FIG. 21C) and CCR9+(FIG. 21E) cells, percentage out of OT-1 cells in respective groups. FIG. 21E shows a Western blot confirming successful knockout of AHR, with β-actin as control. Ctrl cells: CD45.1− and GPR15 knockout cells: CD45.1+.

FIG. 21F shows expression of a4b7 and GPR15 when mice are feed a regular diet or a Vitamin A deficient diet.

FIG. 22 shows the ratio of [Ctrl] to [AHR knockout] OT-1 cells in tissues at 7 days post immunization, normalized to spleen. Consistent with the level of GPR15, at day 7, AHR KO cells were under-represented in the colon and cecum in the 10-CL-BBQ and Am80+10-CL-BBQ groups, but not in the DMSO and Am80 groups.

FIG. 23 shows the ratio of [Ctrl] to [AHR knockout] OT-1 cells in tissues at 7 days post immunization, normalized to spleen. Kruskal-Wallis test (one-way ANOVA) and Dunn's multiple comparisons test (pair-wise with bLN as ctrl). *p<0.05, **p<0.01, ***p<0.001, and ****p<0.0001. ns, not significant.

FIG. 24 shows a schematic parental vaccine with Am80+10-CL-BBQ to assay for gut trafficking.

FIGS. 25A-25C Validation of antibody blockade of α4β7 in the parenteral vaccine setting. OT-1 cells were transferred into B6 mice and recipients were immunized s.c. with OVA+CpG on day 0, and injected with Am80+10-CL-BBQ on day 0, 1 and 2. Either anti-mouse α4β7 blocking antibody or isotype control antibody was given i.p. at 100 ug/100 ul per day on day 3,4,5 and 6. At day 5, blood was analyzed by flow cytometry. Blockade of α4β7 reduced signal intensity of α4β7 on OT-1 cells, but did not alter the percentage of α4β7+ cells. Blockade of α4β7 also altered the presence of CCR9+ cells in the blood, and presence of GPR15+ cells (not significant). FIG. 25A Blood at day 5, representative flow cytometry plots gated on OT-1 cells. OT-1 cells in the anti-α4β7 treated mice showed lower staining of α4β7 (using the same antibody clone). FIG. 25B Blood at day 5. gMFI of α4β7 on OT-1 cells. FIG. 25C shows percentage of OT-1 cells that expressed α4β7, CCR9 and GPR15 in day 5 blood. Welch's t test. *p<0.05, **p<0.01, ***p<0.001, and ****p<0.0001. ns, not significant.

FIG. 26 Antibody blockade of α4β7 reduced OT-1 T cells in the small and large intestine. OT-1 cells were transferred into B6 mice and recipients were immunized s.c. with OVA+CpG on day 0, and injected with Am80+10-CL-BBQ on day 0, 1 and 2. Either anti-mouse α4β7 blocking antibody or isotype control antibody was given i.p. at 100 ug/100 ul per day on day 3,4,5 and 6. At day 7, tissues were harvested and analyzed by flow cytometry. Blockade of α4β7 reduced OT-1 cells in the small and large intestine, without affecting the spleen. Percentage of OT-1 cells in respective tissues at day 7. Mice treated with α4β7 blocking antibody showed lower percentage of OT-1 cells in the small and large intestine. Welch's t test. *p<0.05, **p<0.01, ***p<0.001, and ****p<0.0001. ns, not significant.

FIGS. 27A-27F Characterization of resident memory cells in the small and large intestine. Wildtype B6 recipients were adoptively transferred with 2×104 GFP+OT-1 cells and immunized and treated with the same strategy as described previously. Spleen, small intestine (IEL and LPL), cecum and colon were harvested at 3-4 months and analyzed by flow cytometry. Am80+10-CL-BBQ generated the highest percentage/number of resident memory OT-1 cells in all intestinal tissues, characterized by the expression of CD103 and CD69. Percentage of OT-1 cells out of CD8a+ cells in small intestine IEL (FIG. 27A), small intestine LPL (FIG. 27B), colon (FIG. 27C), and cecum (FIG. 27D). FIG. 27E shows the number of OT-1 cells recovered. FIG. 27G shows expression of CD69 and CD103 in intestinal tissue resident OT-1 cells generated by combined treatment of Am80+10-CL-BBQ. Brown-Forsythe and Welch's ANOVA with Dunnett's T3 multiple comparisons test. *p<0.05, **p<0.01, ***p<0.001, and ****p<0.0001. ns, not significant.

FIGS. 28A-28D Characterization of resident memory cells in the small and large intestine. Spleens were harvested and analyzed by flow cytometry. Am80+10-CL-BBQ did not impair generation and composition of systemic CD8+ T cell memory responses. FIG. 28A shows the percentage of OT-1 cells out of CD8a+ cells. FIG. 28B shows the number of total OT-1 cells recovered. FIG. 28C shows a representative flow cytometry plot of circulating memory cells subsets (Tcm-central memory, Tpm-peripheral memory, and Tem-effector memory cells) as defined by expression of CXCR3 and CX3CR1. Data was from spleen of an Am80+10-CL-BBQ treated mouse, gated on OT-1 cells. FIG. 28D shows the percentage of each circulating memory cell subset out to total OT-1 cells in the spleen. Brown-Forsythe and Welch's ANOVA with Dunnett's T3 multiple comparisons test. *p<0.05, **p<0.01, ***p<0.001, and ****p<0.0001. ns, not significant.

FIG. 29 depicts a schematic for intra-rectal Listeria infection model. Female BALB/c mice were immunized subcutaneously with 50 ug LLO91-99 peptide+CpG-1668 on day 0, and injected with DMSO, Am80, 10-CL-BBQ or Am80+10-CL-BBQ on day 0, 1 and 2. Six weeks post vaccination, mice were fasted overnight, and the next day inoculated intra-rectally with 4×109 CFU Listeria. For the infection, mice were fasted overnight, anesthetized with ketamine+xylazine, and the inoculum was slowly delivered in 20 μl volume. Mice were monitored daily, and colon and spleen were harvested 3 days post infection, weighed and plated on BHI agar plates for CFU count.

FIGS. 30A-30D Bacterial Burden following intra-rectal Listeria challenge. Compared to DMSO, Am80 and 10-CL-BBQ groups, mice treated with Am80+10-CL-BBQ showed lower bacterial burden in the colon and spleen, indicative of stronger vaccine protection. FIG. 30A shows colony forming units (CFU) in the spleen, per tissue and (FIG. 30B) per mg tissue. FIG. 30C shows CFU in the colon, per tissue and (FIG. 30D) per mg tissue; Kruskal-Wallis test with Dunn's multiple comparison. *p<0.05, **p<0.01, ***p<0.001, and ****p<0.0001. ns, not significant.

FIGS. 31A-31D Weight loss following intra-rectal Listeria challenge. Compared to DMSO, Am80 and 10-CL-BBQ groups, mice treated with Am80+10-CL-BBQ showed less weight loss, indicative of stronger protection against infection/disease symptoms. (FIG. 31A) Weight curve (percentage of starting weight); percentage body weight at day 2 (FIG. 31B) and day 3 (FIG. 31C) following infection; (FIG. 31D) number of mice reaching humane endpoint at day 3 (weight loss>=20%). Kruskal-Wallis test with Dunn's multiple comparison. *p<0.05, **p<0.01, ***p<0.001, and ****p<0.0001. ns, not significant.

### DETAILED DESCRIPTION

Disclosed herein is a method for modulating adaptive immune responses in the colon and small intestine by promoting or inhibiting lymphocyte trafficking from the blood into the colonic mucosa. Specifically, the methods described herein induce increased levels of lymphocytes (e.g., CD8+ T cells) in (e.g., by increasing traffic of lymphocytes to) the small intestine and/or colon by administration of AHR and/or RAR agonists. Alternatively, the methods described herein decreased levels of lymphocytes (e.g., CD8+ T cells) in (e.g., by decreasing traffic of lymphocytes to) the small intestine and/or colon by administration of AHR and/or RAR inhibitors.

The methods disclosed herein can be applied to various clinical settings, such as vaccine development, cancer therapy and treatment of inflammatory bowel disease. For example, a parenteral model vaccine can contain select immune modulators to simultaneously stimulate two signaling pathways, via retinoic acid receptors (RAR) and aryl hydrocarbon receptors (AHR), promoting the acquisition of colon homing properties (a phenomenon known as ‘imprinting’) by antigen-specific activated lymphocytes. In another example, RAR and AHR inhibitors can decrease antigen-specific activated lymphocytes in the colon and/or small intestine.

### Definitions

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

An “effective amount” or “therapeutically effective amount” of a compound is that amount of a compound which is sufficient to provide a beneficial effect to the subject to which the compound is administered.

A “therapeutic” treatment is a treatment administered to a subject who exhibits signs or symptoms of pathology, for the purpose of diminishing or eliminating those signs or symptoms.

As used herein, “treating a disease or disorder” means reducing the severity and/or frequency with which a sign or symptom of the disease or disorder is experienced by a subject. Disease and disorder are used interchangeably herein.

The terms “increased,” “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a control level, or at least about a 2-fold, or at least about a 3-fold, or at least about a 4-fold, or at least about a 5-fold or at least about a 10-fold increase, or any increase between 2-fold and 10-fold or greater as compared to a control level.

The terms “decreased,” “decrease” or “inhibit” are all used herein to generally mean a decrease by a statically significant amount; for the avoidance of any doubt, the terms “decreased,” “decrease” or “inhibit” means a decrease of at least 10% as compared to a reference level, for example a decrease of at least about 20%, or at least about 30%, or at least about 40%, or at least about 50%, or at least about 60%, or at least about 70%, or at least about 80%, or at least about 90% or up to and including a 100% decrease or any decrease between 10-100% as compared to a control level, or at least about a 2-fold, or at least about a 3-fold, or at least about a 4-fold, or at least about a 5-fold or at least about a 10-fold decrease, or any decrease between 2-fold and 10-fold or greater as compared to a control level.

### Lymphocytes

Lymphocytes are a type of white blood cell involved in immune system regulation. Lymphocytes are much more common in the lymphatic system, and include B cells, T cells, killer T-cells, and natural killer (NK) cells. Two broad classes of lymphocytes are recognized: the B-lymphocytes (B-cells), which are precursors of antibody-secreting cells, and T-lymphocytes (T-cells).

The terms “T cell” or “T lymphocyte” are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. Full responsiveness of a T cell requires, in addition to receptor engagement, an accessory cell-delivered costimulatory activity, e.g., engagement of CD28 on the T cell by CD80 and/or CD86 on the antigen presenting cell (APC).

T-cells are subdivided into two distinct classes based on the cell surface receptors they express. The majority of T cells express T cell receptors (TCR) consisting of α and β-chains. A small group of T cells express receptors made of 7 and 6 chains. Among the α/β T cells are two sub-lineages: those that express the coreceptor molecule CD4 (CD4+ T cells); and those that express CD8 (CD8+ T cells). These cells differ in how they recognize antigen and in their effector and regulatory functions.

CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated.

CD8+(cytotoxic) T cells, like CD4+ Helper T cells, are generated in the thymus and express the T-cell receptor. However, rather than the CD4 molecule, cytotoxic T cells express a dimeric co-receptor, CD8, usually composed of one CD8α and one CD8β chain. CD8+ T cells (often called cytotoxic T lymphocytes, or CTLs) are important for immune defense against intracellular pathogens, including viruses and bacteria, and for tumor surveillance. Additionally, activated CD8+ T cells express FasL on the cell surface, which binds to its receptor, Fas, on the surface of the target cell. These signaling molecules result in the activation of the caspase cascade, which also results in apoptosis of the target cell.

Memory T cells provide long lasting protection once the immune system first encounters an antigen, e.g., a virus, bacteria, cancer cell, or inflammation. Memory T cells include central memory T cells (Tcm), effector memory T cells (Tem), and peripheral memory T cells (Tpm). Tcm reside in lymphoid tissues and maintain the memory T cell pool. Tcm can circulate, but are mostly found in the lymph nodes. Tem are capable of producing cytokines and carrying out effector functions. Tem circulate through the body and can rapidly respond to re-encounter with the antigen. Tpm are a subset of memory T cells that reside in peripheral tissues like the skin, gut, and lungs, rather than in lymph nodes.

Circulating memory T cells are a population of T lymphocytes that persist in the bloodstream after an infection is cleared, providing long-term protection against reinfection. Circulating memory T cells can express CXCR3, which is involves with recruitment of the T cells to sites of inflammation. CX3CR1 can also be expressed by circulating memory cells for localization of T cells within tissues.

As used herein, the term a “disease or disorder of the colon or small intestine” refers to a disease or disorder that effects the small intestine or colon. The colon and small intestine are the lower part of the gastrointestinal (GI) tract, or “gut”. The GI tract includes the mouth, esophagus, stomach, small intestine, which includes intraepithelial lymphocytes (IEL) and lamina propria lymphocytes (LPL), and large intestine, which includes the cecum, proximal colon, mid colon, distal colon.

The disease or disorder of the colon or small intestine can be a cancer, an autoimmune disease, or an enteric pathogenic infection. The cancer is colorectal cancer, adenocarcinoma, sarcoma, carcinoid tumor, gastrointestinal stromal tumor (GIST), or intestinal lymphoma. The autoimmune disease is Crohn's disease, ulcerative colitis, celiac disease, autoimmune enteropathy, eosinophilic colitis, Behcet's disease, or autoimmune gastritis. The enteric pathogenic infection can be bacterial, viral, or parasites. Bacterial infections can be from Salmonella, Shigella, Clostidioides difficile, Campylobacter jejuni, Vibrio cholera, Yersinia enterocolitica, Escherichia coli, or Listeria monocytogenes. In one embodiment, the enteric pathogenic infection is Listeria monocytogenes. Viral infections can be rotavirus, poliovirus, adenovirus, or norovirus. Pathogenic parasites can be Giardia or Cryptosporidium.

In one embodiment, the term “control” or “control level” refers to a level of, for example, CD8+, GPR15, or α4β7, in a sample or a subject prior to the administering a treatment, e.g., RAR agonist and/or AHR agonist with or without a therapeutic agent. The control is used to compare with the level in a sample derived from the subject or in a subject after administering the treatment, e.g., RAR agonist and/or AHR agonist with or without a therapeutic agent. In one embodiment, the control is based on the level of, e.g., CD8+, GPR15, or α4β7, from a subject having a disease or disorder of the colon and/or small intestine. In a particular embodiment, the control level refers to the level of CD8+ T cells in the small intestine or colon of a subject prior to administration of the RAR agonist and/or AHR agonist, optionally with a therapeutic agent.

In another embodiment, the control is a standardized control, such as, for example, a control which is predetermined using an average of the levels of CD8+ T cells in the colon or small intestine prior to treatment with RAR and/or AHR agonists and/or therapeutic agent. In some embodiments, the average levels of CD8+ T cells from a population of subjects having a disease or disorder of the colon and/or small intestine. In some embodiments, the average levels of CD8+ T cells from a population of healthy subjects.

In still other embodiments of the invention, a control level is predetermined using an average of the levels of CD8+ T cells in the colon or small intestine prior to treatment with RAR and/or AHR inhibitors and/or therapeutic agent. In some embodiments, the average levels of CD8+ T cells is from a population of subjects having a disease or disorder of the colon and/or small intestine. In some embodiments, the average levels of CD8+ T cells are from a population of healthy subjects.

### Methods of Treatment

In some embodiments of the methods provided herein include administering to a subject in need thereof a therapeutically effective amount of a retinoic acid receptor (RAR) agonist and/or an aryl hydrocarbon receptor (AHR) agonist thereby increasing CD8+ T cells in the colon and/or small intestine compared to a control. In some embodiments, the method further comprises administering a therapeutic agent. Increasing CD8+ T-cells after administration of the RAR agonists and/or the AHR agonists, and/or therapeutic agents results in the induction of proliferation and/or accumulation of CD8+ T-cells in the colon and/or small intestine in the subject.

In other embodiments of the methods provided herein include administering to a subject in need thereof a therapeutically effective amount of a retinoic acid receptor (RAR) agonist and/or an aryl hydrocarbon receptor (AHR) agonist, thereby increasing trafficking of CD8+ T cells to the colon and/or small intestine compared to a control. In some embodiments, the method further comprises administering a therapeutic agent. Increasing trafficking of CD8+ T-cells after administration of the RAR agonists and/or the AHR agonists, and/or therapeutic agents results in the accumulation of CD8+ T-cells in the colon and/or small intestine in the subject.

In some embodiments, the method can increase CD8+ T cells between about 5% to about 70%, about 10% to about 50%, about 20% to about 30%, about 55% to about 65%, or about 15% to about 40% in the colon and/or small intestine compared to a control. In some embodiments, the CD8+ T cells can increase about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 70%, about 30% to about 65%, about 30% to about 60%, about 30% to about 55%, about 30% to about 50%, about 30% to about 45%, about 30% to about 40%, about 30% to about 35%, about 35% to about 70%, about 35% to about 65%, about 35% to about 60%, about 35% to about 55%, about 35% to about 50%, about 35% to about 45%, about 35% to about 40%, about 40% to about 70%, about 40% to about 65%, about 40% to about 60%, about 40% to about 55%, about 40% to about 50%, about 40% to about 45%, about 45% to about 70%, about 45% to about 65%, about 45% to about 60%, about 45% to about 55%, about 45% to about 50%, about 50% to about 70%, about 50% to about 65%, about 50% to about 60%, about 50% to about 55%, about 55% to about 70%, about 55% to about 65%, about 55% to about 60%, about 60% to about 70%, about 60% to about 65%, or about 65% to about 70% in the colon and/or small intestine compared to a control. In some embodiments, the control comprises a level of CD8+ T cells in the colon and/or small intestine prior to the administration of RAR agonists and/or AHR agonists.

In some embodiments, the CD8+ T cells in the colon and/or small intestine can express CD69+ and/or CD103+. In some embodiments, the CD8+ T cells in the colon and/or small intestine express CD69+CD103+. In some embodiments, between about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 100%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 100%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 75% to about 80%, about 80% to about 100%, about 80% to about 95%, about 80% to about 90%, about 80% to about 85%, about 85% to about 100%, about 85% to about 95%, about 85% to about 90%, about 90% to about 100%, about 90% to about 95%, or about 95% to about 100% of the CD8+ T cells in the colon and/or small intestine express CD69+ and/or CD103+.

In some embodiments, the CD8+ T cells in the colon and/or small intestine can express one or more of α4β7, GPR15, and/or CCR9 compared to a control. In some embodiments, the CD8+ T cells in the colon and/or small intestine express one or more of α4β7, GPR15, and CCR9 compared to a control. In some embodiments, between about 20% to about 100%, about 20% to about 95%, about 20% to about 90%, about 20% to about 85%, about 20% to about 80%, about 20% to about 75%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 100%, about 25% to about 95%, about 25% to about 90%, about 25% to about 85%, about 25% to about 80%, about 25% to about 75%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 100%, about 30% to about 95%, about 30% to about 90%, about 30% to about 85%, about 30% to about 80%, about 30% to about 75%, about 30% to about 70%, about 30% to about 65%, about 30% to about 60%, about 30% to about 55%, about 30% to about 50%, about 30% to about 45%, about 30% to about 40%, about 30% to about 35%, about 35% to about 100%, about 35% to about 95%, about 35% to about 90%, about 35% to about 85%, about 35% to about 80%, about 35% to about 75%, about 35% to about 70%, about 35% to about 65%, about 35% to about 60%, about 35% to about 55%, about 35% to about 50%, about 35% to about 45%, about 35% to about 40%, about 40% to about 100%, about 40% to about 95%, about 40% to about 90%, about 40% to about 85%, about 40% to about 80%, about 40% to about 75%, about 40% to about 70%, about 40% to about 65%, about 40% to about 60%, about 40% to about 55%, about 40% to about 50%, about 40% to about 45%, about 45% to about 100%, about 45% to about 95%, about 45% to about 90%, about 45% to about 85%, about 45% to about 80%, about 45% to about 75%, about 45% to about 70%, about 45% to about 65%, about 45% to about 60%, about 45% to about 55%, about 45% to about 50%, about 50% to about 100%, about 50% to about 95%, about 50% to about 90%, about 50% to about 85%, about 50% to about 80%, about 50% to about 75%, about 50% to about 70%, about 50% to about 65%, about 50% to about 60%, about 50% to about 55%, about 55% to about 100%, about 55% to about 95%, about 55% to about 90%, about 55% to about 85%, about 55% to about 80%, about 55% to about 75%, about 55% to about 70%, about 55% to about 65%, about 55% to about 60%, about 60% to about 100%, about 60% to about 95%, about 60% to about 90%, about 60% to about 85%, about 60% to about 80%, about 60% to about 75%, about 60% to about 70%, about 60% to about 65%, about 65% to about 100%, about 65% to about 95%, about 65% to about 90%, about 65% to about 85%, about 65% to about 80%, about 65% to about 75%, about 65% to about 70%, about 70% to about 100%, about 70% to about 95%, about 70% to about 90%, about 70% to about 85%, about 70% to about 80%, about 70% to about 75%, about 75% to about 100%, about 75% to about 95%, about 75% to about 90%, about 75% to about 85%, about 75% to about 80%, about 80% to about 100%, about 80% to about 95%, about 80% to about 90%, about 80% to about 85%, about 85% to about 100%, about 85% to about 95%, about 85% to about 90%, about 90% to about 100%, about 90% to about 95%, or about 95% to about 100% of the CD8+ T cells in the colon and/or small intestine express α4β7, GPR15, and/or CCR9 compared to a control.

In some embodiments, between about 20% to about 100%, about 30% to about 60%, about 40% to about 60%, about 30% to about 70%, about 60% to about 80% of the CD8+ T cells in the colon and/or small intestine express α4β7 compared to a level of CD8+α4β7+ T cells in the colon and/or small intestine prior to the administration of RAR agonists and/or AHR agonists.

In some embodiments, between about 20% to about 100%, about 30% to about 60%, about 40% to about 60%, about 30% to about 70%, about 60% to about 80% of the CD8+ T cells in the colon and/or small intestine express GPR15 compared to a level of CD8+GPR15+ T cells in the colon and/or small intestine prior to the administration of RAR agonists and/or AHR agonists.

In some embodiments, between about 20% to about 100%, about 30% to about 60%, about 40% to about 60%, about 30% to about 70%, about 60% to about 80% of the CD8+ T cells in the colon and/or small intestine express CCR9 compared to a level of CD8+CCR9+ T cells in the colon and/or small intestine prior to the administration of RAR agonists and/or AHR agonists.

In other embodiments of the methods provided herein include administering a retinoic acid receptor (RAR) agonist and/or an aryl hydrocarbon receptor (AHR) agonist before, concomitantly, or after administering the therapeutic agent, thereby increasing the efficacy of the therapeutic agent compared to a control. In some embodiments, the control comprises a level of efficacy of the therapeutic agent prior to the administration of RAR agonists and/or AHR agonists.

In some embodiments, the efficacy of the therapeutic agent can be measured by the level of CD8+ T cells in the colon and/or small intestine after 1 month, 2 months, 6 months, 1 year, 2 years, or 5 years after administering the RAR agonists and/or AHR agonists and therapeutic agent, compared to level of CD8+ T cells in the colon and/or small intestine prior to administering the RAR agonists and/or AHR agonists and therapeutic agent.

In some embodiments, the RAR agonists and/or the AHR agonists described herein can be administered to a subject concomitantly with one or more additional therapeutic agents. In some embodiments, the RAR agonists and/or the AHR agonists can be administered to a subject followed by administration of one or more additional therapeutic agents. In some embodiments, any of the RAR agonists and/or the AHR agonists described herein is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after to administration of the one or more additional therapeutic agents. Alternatively, in some embodiments, one or more therapeutic agents administered to a subject can be followed by administration of any of the RAR agonists and/or the AHR agonists. In some embodiments, one or more therapeutic agents can be administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of any the RAR agonists and/or the AHR agonists.

In another aspect, the methods provided herein are for treating a disease or disorder of the colon and/or small intestine, including administering to a subject in need thereof a therapeutically effective amount of a retinoic acid receptor (RAR) agonist and/or an aryl hydrocarbon receptor (AHR) agonist, and a therapeutic agent, thereby treating disease or disorder of the colon and/or small intestine.

As used herein, “therapeutic agent” refers to a pharmaceutical composition used for prophylaxis or treatment of a disease or disorder of the colon and/or small intestine. The therapeutic agent can include, but are not limited to, a vaccine, anti-inflammatory, chemotherapeutic, probiotic and/or an antibiotic.

As used herein, “vaccine” is as a composition of antigenic moieties, usually consisting of modified-live (attenuated) or inactivated infectious agents, or some part of the infectious agents, that is administered, most often with an adjuvant, into the body to produce active immunity.

An “antigen” is a compound which, when introduced into an animal or a human, will result in the formation of antibodies and cell-mediated immunity. Vaccine can be combined with one or more adjuvant. An “adjuvant” is a compound or compounds that, when used in combination with specific vaccine antigens in formulations, augment or otherwise alter or modify the resultant immune responses.

The term “probiotic” refers to one or more bacteria that can be administered to a subject to aid in the restoration of a subject's microbiota by increasing the number of bacteria that are desired, preferred, neutral, beneficial and/or under-represented in the subject's microbiota.

RAR agonist can be all-trans retinoic acid (ATRA), AM80, AM580, AC 261066, Adapalene, BMS 753, BMS 961, CD 1530, CD2314, CD437, ch 55, DC271, retinoic acid, TTNPB, etretinate, tazarotene, tamibarotene, or a combination thereof. RAR agonists can include, but are not limited to, 4-[(E)-2-(5,6,7,8-Tetrahydro-5,5,8,8-tetramethyl-2-naphthalenyl)-1-propenyl]benzoic acid (TTNPB); 4-[4-(2-Butoxyethoxy-)-5-methyl-2-thiazolyl]-2-fluorobenzoic acid (AC 261066); 6-(4-Methoxy-3-tricyclo[3.3.1.13,7]dec-1-ylphenyl)-2-naphthalenecarboxylic acid (Adapalene); 4-[(5,6,7,8-Tetrahydro-5,5,8,8-tetramethyl-2-naphthalenyl)carboxamido]benzoic acid (Am580); 4-[[(5,6,7,8-Tetrahydro-5,5,8,8-tetramethyl-2-naphthalenyl)amino]carbonyl]benzoic acid (AM80); 4-[[(2,3-Dihydro-1,1,3,3-tetramethyl-2-oxo-1H-inden-5-yl)carbonyl]amino]benzoic acid (BMS 753); 3-Fluoro-4-[[2-hydroxy-2-(5,5,8,8-tetramethyl-5,6,7,8,-tetrahydro-2-naphthalenyl)acetyl]amino]-benzoic acid (BMS 961); 4-(6-Hydroxy-7-tricyclo[3.3.1.13,7]dec-1-yl-2-naphthalenyl)benzoic acid (CD 1530); 5-(5,6,7,8-Tetrahydro-5,5,8,8-tetramethyl-2-anthracenyl)-3-thiophenecarboxylic acid (CD 2314); 6-(4-Hydroxy-3-tricyclo[3.3.1.13,7]dec-1-ylphenyl)-2-naphthalenecarboxylic acid (CD 437); 4-[(1E)-3-[3,5-bis(1,1-Dimethylethyl)phenyl]-3-oxo-1-propenyl]benzoic acid (Ch 55); 4-[2-[1,2,3,4-Tetrahydro-4,4-dimethyl-1-(1-methylethyl)-6-quinolinyl]ethynyl]benzoic acid (DC 271); or 3,7-Dimethyl-9-(2,6,6-trimethyl-1-cyclohexen-1-yl)-2E,4E,6E,8E,-nonatetraenoic acid (retinoic acid).

AHR agonist can be 10-CL-BBQ, L-kynurenine, ITE, FICZ, indirubin, VAF347, a flavonoid, a carotinoid, a glucobrassin metabolite, a tryptophan metabolite, or a combination thereof. AHR agonists can include, but are not limited to, 10-CL-BBQ, L-kynurenine, ITE, FICZ, indirubin, VAF347, other ligands such as flavonoids, carotinoids, glucobrassin metabolites, and tryptophan metabolites. Non-limiting example also include, 3,3-diindolylmethane (DIM); Beta-naphthoflavone; Bilirubin; Biliverdin; Curcumin; Diclofenac; Diosmin; Gallic acid; Hydroxyeicosatrienoic acid ([12(R)-HETE]); Cinnabarinic acid (CA); 5-hydroxytryptophan (5HTP); 4-hydroxy-tamoxifen (4OHT); 6-Methyl-1,3,8-trichlorodibenzofuran (6-MCDF); 3-Methylindole (Skatole); 3′4′-Dimethoxyflavone (DMF); 1,4-dihydroxy-2-naphthoic acid (DHNA); 2-(Indol-3-ylmethyl)-3,39-diindolylmethane (Ltr-1); 5,11-Dihydroindolo[3,2-b]carbazole-6-carboxaldehyde (FICZ); 10-Chloro-7H-benzimidazo[2,1-a]benzl[de]isoquinolin-7-one (10-CL-BBQ); 1-Methyl-N-[2-methyl-4-[2-(2-methylphenyl)diazenyl]phenyl-1H-pyrazole-5-carboxamide (CH 223191); 1-(4-Methylphenyl)-2-(4,5,6,7-tetrahydro-2-imino-3(2H)-benzothiazolyl)ethanone hydrobromide (Pifithrin-α hydrobromide); 2-(1H-Indol-3-ylcarbonyl)-4-thiazolecarboxylic acid methyl ester (ITE); 2,3,7,8-Tetrachlorodibenzo-p-dioxin (TCDD); 3,3′,4,4′,5-Pentachlorobihpenyl; 1,2,3,6,7,8-Hexachlorodibenzo[b,d]furan; Benzo[a]pyrene (BaP); Benzo(k)fluoranthene; 3-Methylcholanthrene (3 MC); Indigo; Indirubin; Indole; Indole-3-acetic acid (IAA); Indole-3-acetonitrile (I3ACN); Indole-3-aldehyde (IAId); Indole-3-carbinol (I3C); Indolo [3,2-b]carbazole (ICZ); Indoxyl-3-sulfate (I3S); Kynurenic acid (KA); 1-Kynurenine (Kyn); Laquinimod; Lipoxin A4; Malassezin; 3′-methoxy-4′-nitroflavone (MNF); Norisoboldine; Omeprazole; Prostaglandin; Quercetin; StemRegenin 1 (SR1); Sulindac; Resveratrol; Tapinarof; Tryptamine; Trypthantrin; VAF347; and Xanthurenic acid.

The RAR agonist and/or AHR agonist, and a therapeutic agent can be administered to a subject to treat a disease or disorder of the colon or small intestine. In some embodiments, the RAR agonist can be administered to the subject. In some embodiments, the AHR agonist can be administered to the subject. In some embodiments, the RAR agonist and AHR agonist can be administered to the subject. In some embodiment, the RAR agonist and/or the AHR agonist are administered before the therapeutic agent.

In some embodiments, administering the RAR agonist results in an increase in the level of CD8+ T cells in the small intestine. In some embodiments, administering the AHR agonist results in an increase in the level of CD8+ T cells in the colon.

In some embodiments, the RAR agonist and/or the AHR agonist and the therapeutic agent are administered concomitantly.

The some embodiments, the RAR agonist and/or the AHR agonist and the therapeutic agent are administered sequentially. In another embodiment, the RAR agonist and the AHR agonist are administered separately.

In some embodiments, the RAR agonist and/or the AHR agonist can be administered after the therapeutic agent. In some embodiments, the RAR agonist and/or the AHR agonist can be administered before the therapeutic agent. In one embodiment, the RAR agonist can be administer prior to the AHR agonist.

In some embodiments, the RAR agonist and/or the AHR agonist can be administered twice a day, once a day, once every two days, once a week, twice a week, once a month, once every two months, or once every six months. In another embodiment, the RAR agonist and/or the AHR agonist can be administered for 1 week to 1 year. The AHR agonist can be administered for more doses than the RAR agonist.

The therapeutic agent can be a vaccine. The RAR agonist and/or the AHR agonist can be administered after the vaccine. The RAR agonist and/or the AHR agonist can be administered before the vaccine. In one embodiment, the RAR agonist can be administer prior to the AHR agonist.

The therapeutic agent can be a chemotherapeutic. The RAR agonist and/or the AHR agonist can be administered after the chemotherapeutic. The RAR agonist and/or the AHR agonist can be administered before the chemotherapeutic. In one embodiment, the RAR agonist can be administer prior to the AHR agonist.

The therapeutic agent can be an anti-inflammatory. The RAR agonist and/or the AHR agonist can be administered after the anti-inflammatory. The RAR agonist and/or the AHR agonist can be administered before the anti-inflammatory. In one embodiment, the RAR agonist can be administer prior to the AHR agonist.

The therapeutic agent can be a probiotic. The RAR agonist and/or the AHR agonist can be administered after the probiotic. The RAR agonist and/or the AHR agonist can be administered before the probiotic. In one embodiment, the RAR agonist can be administer prior to the AHR agonist.

### Inhibit CD8+ T Cells Trafficking to Colon

In other embodiments of the methods provided herein include administering to a subject in need thereof a therapeutically effective amount of a retinoic acid receptor (RAR) inhibitor and/or an aryl hydrocarbon receptor (AHR) inhibitor, thereby decreasing the level CD8+ T cells in the colon and/or small intestine compared to a control.

In other embodiments of the methods provided herein include administering to a subject in need thereof a therapeutically effective amount of a retinoic acid receptor (RAR) inhibitor and/or an aryl hydrocarbon receptor (AHR) inhibitor, thereby decreasing trafficking of CD8+ T cells to the colon and/or small intestine compared to a control.

In other embodiments of the methods provided herein include administering to a subject in need thereof a therapeutically effective amount of a retinoic acid receptor (RAR) inhibitor and/or an aryl hydrocarbon receptor (AHR) inhibitor, thereby treating the inflammatory disease or disorder of the colon and/or small intestine.

In some embodiments, the methods provide herein can further comprise administering to the subject a therapeutic agent.

In some embodiments, the control comprises a level of CD8+ T cells in the colon and/or small intestine prior to the administration of the RAR inhibitor and/or the AHR inhibitor.

In some embodiments, the inflammatory disease or disorder of the colon or small intestine an autoimmune disease.

In some embodiments of the methods provided herein include administering to a subject in need thereof a therapeutically effective amount of a retinoic acid receptor (RAR) inhibitor and/or an aryl hydrocarbon receptor (AHR) inhibitor, thereby decreasing CD8+ T cells in the colon and/or small intestine compared to a control. In some embodiments, the method further comprises administering a therapeutic agent. Decreasing CD8+ T-cells after administration of the RAR inhibitors and/or the AHR inhibitors, and/or therapeutic agents results in the reduction of CD8+ T-cells in the colon and/or small intestine in the subject.

In other embodiments of the methods provided herein include administering to a subject in need thereof a therapeutically effective amount of a retinoic acid receptor (RAR) inhibitor and/or an aryl hydrocarbon receptor (AHR) inhibitor, thereby decreasing trafficking of CD8+ T cells to the colon and/or small intestine compared to a control. In some embodiments, the method further comprises administering a therapeutic agent. Decreasing trafficking of CD8+ T-cells after administration of the RAR inhibitors and/or the AHR inhibitors, and/or therapeutic agents results in the reduction of CD8+ T-cells in the colon and/or small intestine in the subject.

In some embodiments, the method can decrease CD8+ T cells between about 5% to about 70%, about 10% to about 50%, about 20% to about 30%, about 55% to about 65%, or about 15% to about 40% in the colon and/or small intestine compared to a control. In some embodiments, the method can decrease CD8+ T cells between about 5% to about 70%, about 5% to about 65%, about 5% to about 60%, about 5% to about 55%, about 5% to about 50%, about 5% to about 45%, about 5% to about 40%, about 5% to about 35%, about 5% to about 30%, about 5% to about 25%, about 5% to about 20%, about 5% to about 15%, about 5% to about 10%, about 10% to about 70%, about 10% to about 65%, about 10% to about 60%, about 10% to about 55%, about 10% to about 50%, about 10% to about 45%, about 10% to about 40%, about 10% to about 35%, about 10% to about 30%, about 10% to about 25%, about 10% to about 20%, about 10% to about 15%, about 15% to about 70%, about 15% to about 65%, about 15% to about 60%, about 15% to about 55%, about 15% to about 50%, about 15% to about 45%, about 15% to about 40%, about 15% to about 35%, about 15% to about 30%, about 15% to about 25%, about 15% to about 20%, about 20% to about 70%, about 20% to about 65%, about 20% to about 60%, about 20% to about 55%, about 20% to about 50%, about 20% to about 45%, about 20% to about 40%, about 20% to about 35%, about 20% to about 30%, about 20% to about 25%, about 25% to about 70%, about 25% to about 65%, about 25% to about 60%, about 25% to about 55%, about 25% to about 50%, about 25% to about 45%, about 25% to about 40%, about 25% to about 35%, about 25% to about 30%, about 30% to about 70%, about 30% to about 65%, about 30% to about 60%, about 30% to about 55%, about 30% to about 50%, about 30% to about 45%, about 30% to about 40%, about 30% to about 35%, about 35% to about 70%, about 35% to about 65%, about 35% to about 60%, about 35% to about 55%, about 35% to about 50%, about 35% to about 45%, about 35% to about 40%, about 40% to about 70%, about 40% to about 65%, about 40% to about 60%, about 40% to about 55%, about 40% to about 50%, about 40% to about 45%, about 45% to about 70%, about 45% to about 65%, about 45% to about 60%, about 45% to about 55%, about 45% to about 50%, about 50% to about 70%, about 50% to about 65%, about 50% to about 60%, about 50% to about 55%, about 55% to about 70%, about 55% to about 65%, about 55% to about 60%, about 60% to about 70%, about 60% to about 65%, or about 65% to about 70% in the colon and/or small intestine compared to a control. In some embodiments, the control comprises a level of CD8+ T cells in the colon and/or small intestine prior to the administration of RAR inhibitors and/or AHR inhibitors.

In some embodiments, the RAR inhibitors and/or the AHR inhibitors described herein can be administered to a subject concomitantly with one or more additional therapeutic agents.

In some embodiments, the RAR inhibitors and/or the AHR inhibitors can be administered to a subject followed by administration of one or more additional therapeutic agents. In some embodiments, any of the RAR inhibitors and/or the AHR inhibitors described herein is administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more after to administration of the one or more additional therapeutic agents. Alternatively, in some embodiments, one or more therapeutic agents administered to a subject can be followed by administration of any of the RAR inhibitors and/or the AHR inhibitors. In some embodiments, one or more therapeutic agents can be administered at least about 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months or more prior to administration of any the RAR inhibitors and/or the AHR inhibitors.

In another aspect, the methods provided herein are for treating a disease or disorder of the colon and/or small intestine, including administering to a subject in need thereof a therapeutically effective amount of a retinoic acid receptor (RAR) inhibitor and/or an aryl hydrocarbon receptor (AHR) inhibitor, and a therapeutic agent, thereby treating disease or disorder of the colon and/or small intestine.

In some embodiments, the RAR inhibitor is selected from the group consisting of 4-[(1E)-2-[5,6-Dihydro-5,5-dimethyl-8-(phenylethynyl)-2-naphthalenyl]ethenyl]-benzoic acid (BMS 493), 4-[2-(5,6,7,8-Tetrahydro-5,5,8,8-tetramethyl-2-naphthalenyl)ethynyl)-benzoic acid (EC 23), AGN 193109-d7,4-[2-[5,6-Dihydro-5,5-dimethyl-8-(4-methylphenyl)-2-naphthalenyl]ethynyl]benzoic Acid Sodium Salt (AGN 193109 Sodium Salt), 4-[[[5,6-Dihydro-5,5-dimethyl-8-(3-quinolinyl)-2-naphthalenyl]carbonyl]amino]benzoic acid (BMS 1695614), 4-[6-[(2-Methoxyethoxy)methoxy]-7-tricyclo[3.3.1.13,7]dec-1-yl-2-naphthalenyl)benzoic acid (CD2665), 4-[5-[8-(1-Methylethyl)-4-phenyl-2-quinolinyl]-1H-pyrrolo-2-benzoic acid (ER50891), 4-(7,8,9,10-Tetrahydro-5,7,7,10,10-pentamethyl-5H-benzo[e]naphtho[2,3-b][1,4]diazepin-13-yl)benzoic acid (LE135), 4-[5-[3,5-Bis(1,1-dimethylethyl)phenyl]-1-[4-[(4-methyl-1-piperazinyl)carbonyl]phenyl]-1H-pyrazol-3-yl]benzoic acid (LY2955303), 6-[2-(5,6,7,8-Tetrahydro-5,5,8,8-tetramethyl-2-naphthalenyl)-1,3-dithiolan-2-yl]-2-naphthalenecarboxylic acid (MM11253), UVI3003, and a combination thereof.

In some embodiments, the AHR inhibitor is selected from the group consisting of (S)-6-(4-chlorophenyl)-N-(1-hydroxypropan-2-yl)-2-(1-methyl-1H-pyrazol-4-yl)-3-oxo-2,3-dihydropyridazine-4-carboxamide (BAY 2416964), (R)-N-(2-(5-fluoropyridin-3-yl)-8-isopropylpyrazolo[1,5-a][1,3,5]triazin-4-yl)-2,3,4,9-tetrahydro-1H-carbazol-3-amine (IK-175), N-(2-(1H-indol-3-yl)ethyl)-5-(5-fluoropyridin-3-yl)-3-methylpyrazolo[1,5-a]pyrimidin-7-amine (KYN-101), (1S,2S)-2-(3,4-dichlorobenzoyl)cyclopropane-1-carboxylic acid (UPF-648), brevifolincarboxylic acid, 26-Deoxyactein, hCYP1B1-IN-2, AHR-IN-1,1,2,3,4,7,8,9-Heptachlorodibenzofuran (1,2,3,4,7,8,9-HpCDF), 1,2,3,4,7,8-Hexachlorodibenzofuran (1,2,3,4,7,8-HxCDF), and a combination thereof.

### Pharmaceutical Composition for Administration

In some embodiments, the RAR agonist and/or the AHR agonist are encapsulated in a nanoparticle comprising one or more polymers.

The one or more polymers can be polylactic-co-glycolic acid (PLGA), polyethylene glycol (PEG), poly ε-caprolactone (PCL), poly lactic acid (PLA), chitosan, dextran, polyvinyl alcohol (PVA), acetylated-dextran (AcDex), or a combination thereof. In one embodiment, the one or more polymers are AcDex.

The term “pharmaceutical composition” is used herein to refer to a composition that is employed to prevent, reduce in intensity, cure or otherwise treat a target condition or disease.

In some embodiments, the RAR agonist and/or the AHR agonist, or the RAR agonist and/or the AHR agonist encapsulated in a nanoparticle are in a formulation comprising a pharmaceutically acceptable carrier. The term “pharmaceutically acceptable carrier” as used herein refers to any substantially non-toxic carrier useable for formulation and administration of the composition of the described invention in which the product of the described invention will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

In some embodiments, the RAR agonist and/or the AHR agonist, or the RAR agonist and/or the AHR agonist encapsulated in a nanoparticle are formulated for subcutaneous injection.

### EXAMPLES

### Example 1: Methods

Animal Study: Adult C57BL/6 mice and BALB/c mice were obtained from Jackson Laboratories. OT-1 β-actin GFP mice were bred by crossing β-actin GFP and OT-1 strains, obtained from Jackson Laboratories.

OT-1 in vivo Assay (Parenteral Immunization): Mice were adoptively transferred with OT-1 cells, and subsequently immunized with 100 g chicken ovalbumin (OVA) and an adjuvant CpG-1668 (0.5 nmol). On day 0.1 and 2, mice were injected s.c. with solvent control (DMSO, 10% v/v in PEG-400), retinoic acid receptor agonist Am80 (500 nmol in PEG-400), AHR agonist 10-CL-BBQ (164 nmol in PEG-400), or Am80+10-CL-BBQ (same dose). At timepoints defined by individual experiments, tissues were harvested and analyzed.

Preparing intestinal samples for flow cytometry: Small intestine (duodenum to ileum) was dissected out and flushed. Peyer's patches and fat were removed, and the tissue was cut into 4-5 pieces, and flipped to expose the luminal surface. Colon and cecum were dissected out, flushed and cut open longitudinally.

Cell Count and Flow Cytometry and Cell Count: For cell counting, samples were diluted and counted on a BD Accuri C6 Plus cytometer. For flow cytometry, samples were run on a Beckman CytoFLEX cytometer, and analysis was done using Flow Jo software V10.

OT-1 in vitro Culture: OT-1 splenocytes were pulsed for 1 hour with 1 μg OVA peptide (257-264) and cultured overnight in complete T cell (RPMI) media. The next day, CD8+ T cells were isolated using mouse CD8a+ T Cell Isolation Kit (Miltenyi) and cultured with various treatment conditions.

Short-term Competitive Homing: OT-1 cells, either in vitro or in vivo generated, were distinguished by congenic markers CD45.1/CD45.2, mixed at around 1:1 ratio and adoptively transferred into recipient animals. At 20-24 hours post transfer, mice were sacrificed, and tissues were harvested for analysis. Ratio of transferred cells were obtained, and homing index was calculated.

CRISPR Knockout of AHR and GPR15: Naïve GFP+OT-1 cells were isolated with mouse CD8a+ T Cell Isolation Kit (Miltenyi) and nucleofected with Cas9 protein, complexed with either control (CD19) gRNA, or GPR15 gRNA. gRNAs were designed using Synthego online tool and synthesized by Synthego. The procedure was done using a Lonza nucleofector, with the P3 Primary cell Kit. Cells were distinguished based on expression of congenic marker CD45.1 and co-transferred into recipient mice. Recipient mice were immunized, and tissues were analyzed at different timepoints.

Western Blot for AHR: In vitro expanded cells were harvested, and protein contents were extracted using RIPA buffer+1% SDS. Western blot was performed according to standard protocol, with anti-mouse AHR antibody at 1:200 (Biolegend #162703). B-actin was used as internal control.

In vivo Antibody Blockade: Mice were adoptively transferred, immunized, and treated as described in “OT-1 in vivo assay”. Either anti-mouse α4β7 blocking antibody or isotype control antibody was given i.p. at 100 ug/100 ul per day on day 3,4,5 and 6. At day 7, tissues were harvested and analyzed by flow cytometry.

Intra-rectal Listeria Challenge: Female BALB/c mice were immunized subcutaneously with 50 ug LLO91-99 peptide+CpG-1668 on day 0, and injected with DMSO, Am80, 10-CL-BBQ or Am80+10-CL-BBQ on day 0, 1 and 2. Six weeks post vaccination, mice were fasted overnight, and the next day inoculated intra-rectally with 4×10⁹ CFU Listeria. For the infection, mice were anesthetized with ketamine+xylazine, and the inoculum was slowly delivered in 201 volume. Mice were monitored daily, and colon and spleen were harvested 3 days post infection, weighed, and plated on BHI agar plates for CFU count.

### Example 2: Targeting CD8+ Lymphocytes to the Small and Large Intestine

The gut immune system comes into frequent contact with dietary compounds, as well as host and microbiota derived metabolites The immunoregulatory roles of many metabolites remain to be understood Additionally, the small and large intestine differ greatly in nutrients, microbiota and metabolite composition, yet how this affects various aspects of immunity in the respective segments is not fully explored.

CD8+ T cell trafficking, or the entry of activated CD8+ T cells from the circulation into the peripheral tissues was examined. Dietary metabolites, such as RAR and AHR agonists, regulate differential CD8+ T cell trafficking to the small intestine or the large intestine.

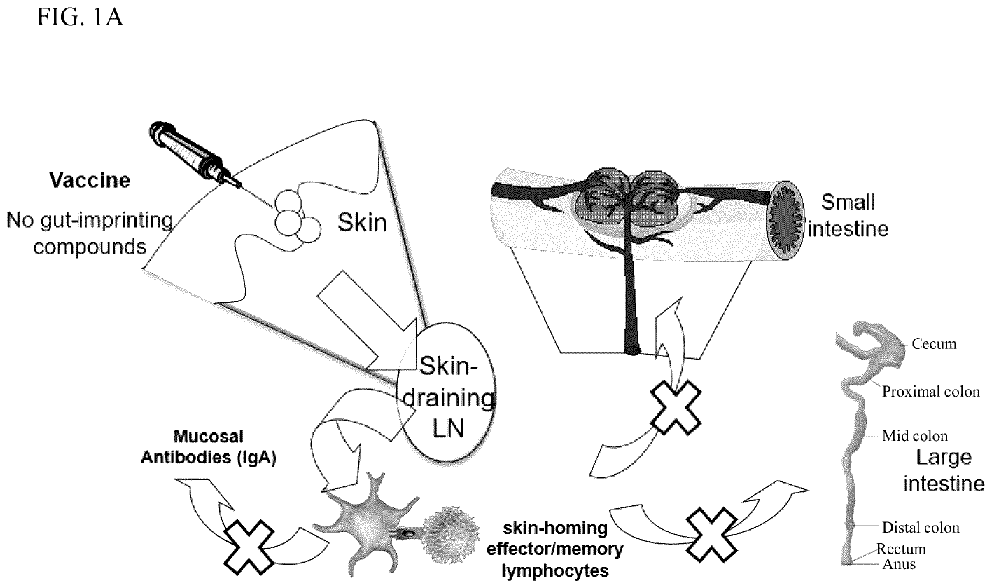

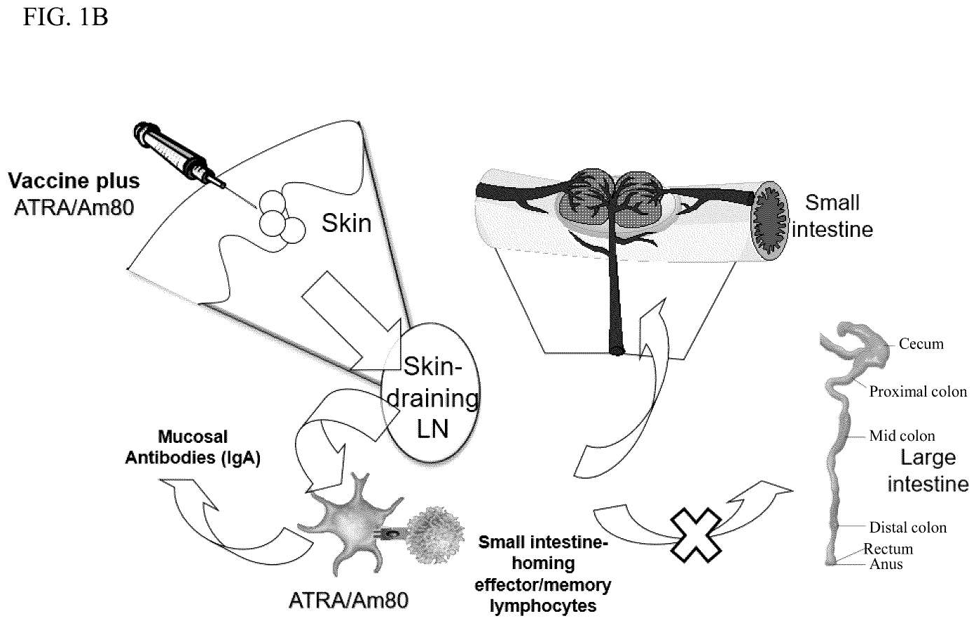

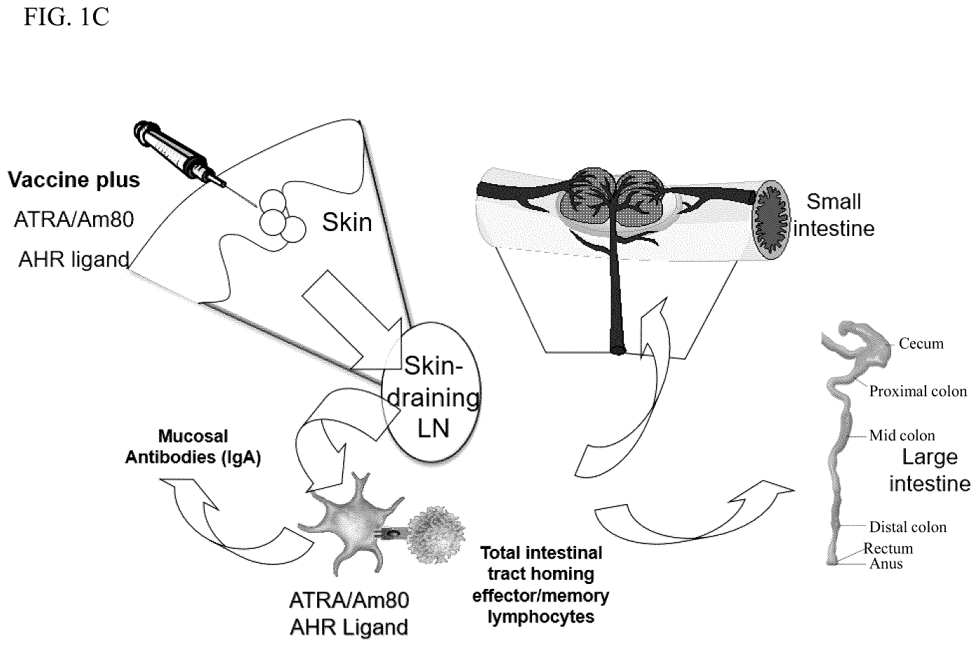

Mice were immunized subcutaneously in combination with synthetic agonists that target molecular sensors of specific metabolic pathways. FIGS. 1A-1C depicts a schematic of parenteral vaccination to target CD8+ lymphocytes to the small and large intestine for total intestinal tract protection. FIG. 1A shows no gut imprinting. FIG. 1B shows treatment with ATRA vaccine and Am80 resulting in production of mucosal antibodies and targeting CD8+T lymphocytes to the small intestine. FIG. 1C shows treatment with ATRA vaccine, Am80 and an AHR ligand resulting in production of mucosal antibodies and targeting CD8+ T lymphocytes to the small and large intestine.

Specifically, retinoic acid, a Vitamin A metabolite, signals through the retinoic acid receptor to up regulate lymphocyte trafficking molecules α4β7 and CCR9 and strongly targets activated T and B cell trafficking to the small, but not the large intestine. In the subcutaneously vaccination model, supplementation with all trans retinoic acid or AM80 (synthetic RAR agonist) increased α4β7 and CCR9 expression, increased CD8+ T cell trafficking to the small intestine, and promoted tissue residence. Conversely, Vitamin A deficiency almost fully abolished the expression of α4β7 on activated CD8+ T cells and resulted in impaired immune responses.

As AM80 was not sufficient for optimal trafficking to the large intestine, other pathways that can enhance colon trafficking were tested. To this end, compounds that achieved substantial cecum and colon targeting, involving the aryl hydrocarbon receptor (AHR) pathway were identified. In conclusion, the parenteral administration of metabolic signaling modulators can enhance gut tropism of CD8+ T cells and is a potential strategy to promote gut protection of parenteral vaccines.

### Objectives

1. To study whether vaccine adjuvantation with retinoic acid receptor agonists target CD8+ T cell responses to the small intestine.

2. To study the role of AHR on targeting CD8+ T cells to the large intestine.

3. To develop an adjuvantation strategy that enhances vaccine responses in the entire intestinal tract.

### Results

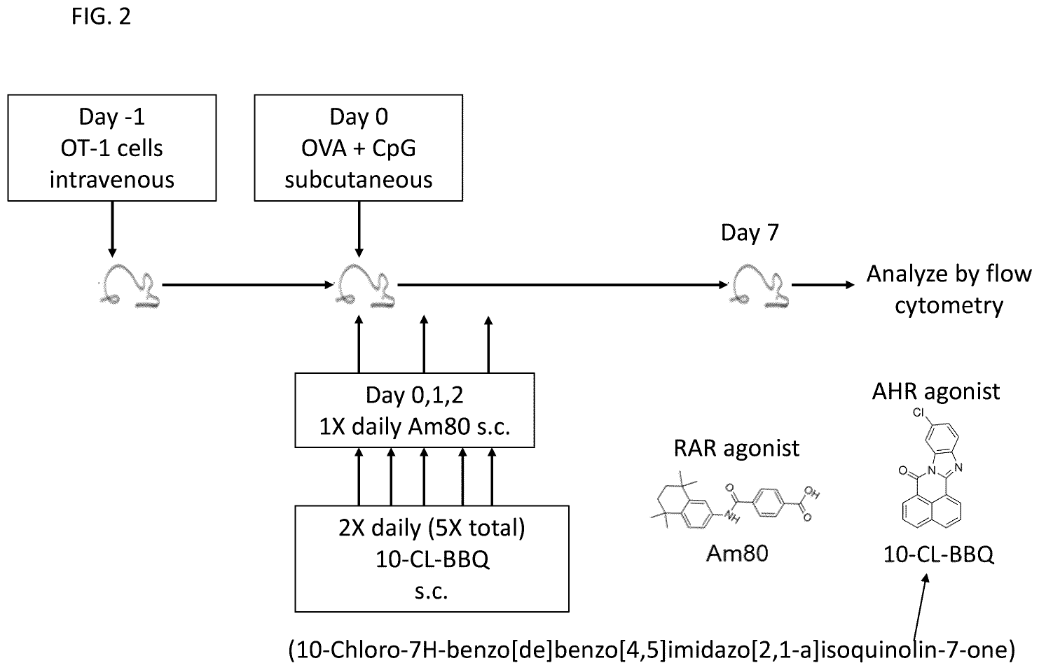

The experimental approach is shown in the schematic in FIG. 2. Antigen-specific CD8+ T cells (OT-1 cells, recognizing a peptide within the chicken ovalbumin protein) were adoptively transferred (1×10⁵ cells) to B6 recipient mice on day −1, and B6 recipients were vaccinated subcutaneously (s.c.) on day 0 with 100 μg chicken ovalbumin (OVA) and an adjuvant CpG-1668 (0.5 nmol). On day 0.1 and 2, mice were injected subcutaneously with solvent control (DMSO, 10% v/v in PEG-400), retinoic acid receptor agonist Am80 (500 nmol in PEG-400), AHR agonist 10-CL-BBQ (164 nmol in PEG-400), or Am80+10-CL-BBQ (same dose). Tissues were harvest on day 7 and analyzed by flow cytometry.

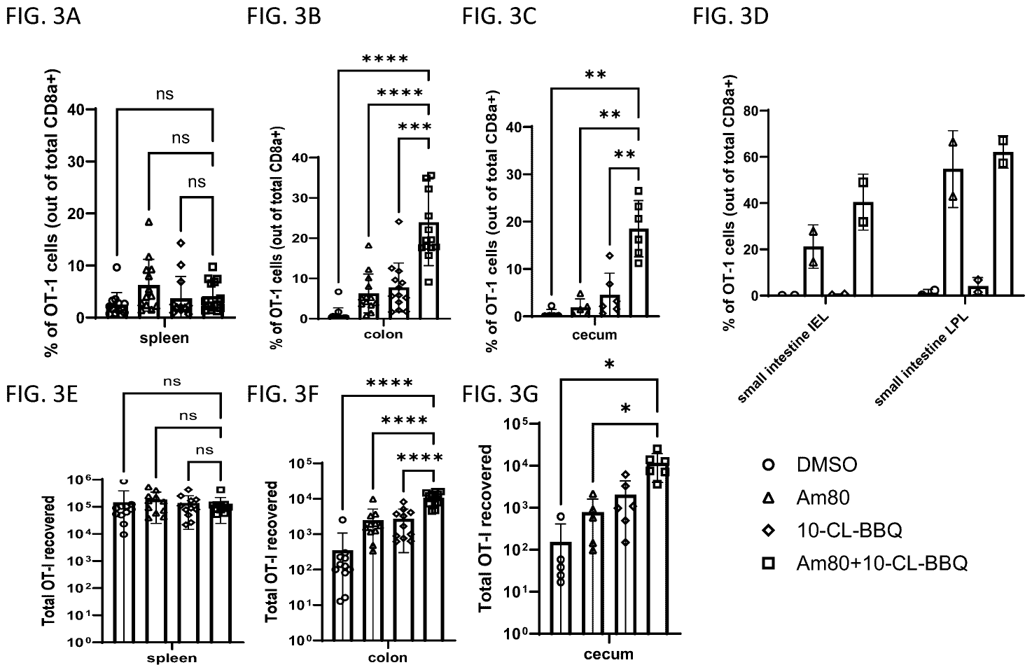

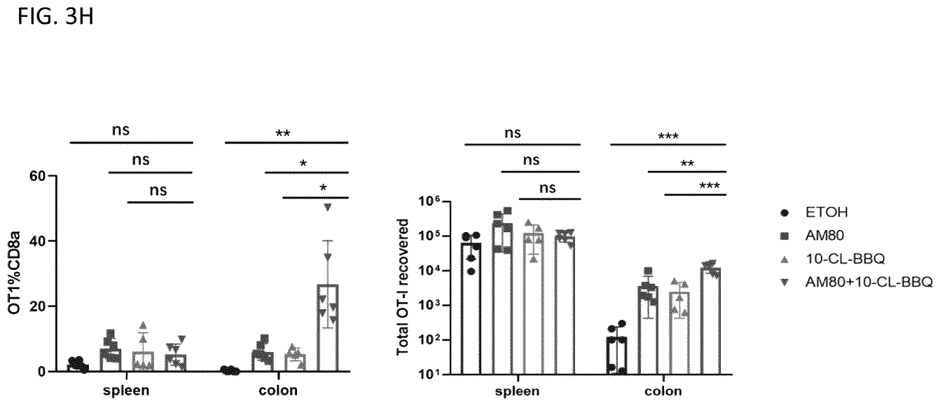

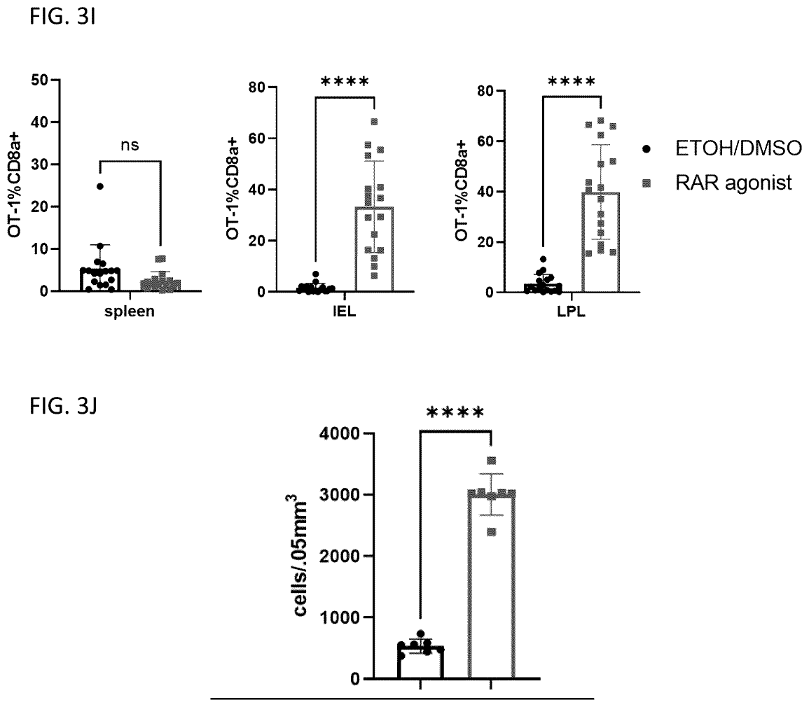

While Am80 or 10-CL-BBQ alone induced some increase of OT-1 effector cells in the colon and cecum, combination of Am80+10-CL-BBQ were the most effective at increasing effector T cells responses in the colon and cecum. Either Am80 alone or Am80+10-CL-BBQ increased OT-1 cells in the small intestine, whereas 10-CL-BBQ alone had no effect. The effects observed in the intestinal tissues were not due to overall higher immune response, as the number and percentage of OT-1 cells in the spleen were similar across all treatment conditions. FIGS. 3A-3D show the percentage of OT-1 cells out of total CD8a+ T cells in the spleen, colon, cecum and small intestine, respectively. FIGS. 3E-3F show the total number of OT-1 cells recovered from the spleen, colon and cecum. Both Brown-Forsythe and Welch's ANOVA with Dunnett's T3 multiple comparisons test were used to generate the p values. *p<0.05, **p<0.01, ***p<0.001, and ****p<0.0001. ns, not significant.

AM80 and 10-Cl-BBQ synergistically targeted OT-I T cells to the colon. Wildtype C57B6 recipient mice were adoptively transferred with OT-I cells from CD45.1+ congenic donors, and 6 hours later all recipients were immunized subcutaneously (scruff) with OVA and CpG (day 0). On day 0, 1 and 2, mice were injected subcutaneously (scruff) with Am80, 10-CL-BBQ, AM80+10-CL-BBQ or ethanol (negative control). AM80 is a retinoic acid receptor agonist and was injected once daily, stocked in ethanol, and formulated in PEG-400 to a final dose of 175 ug/100 ul. 10-CL-BBQ is an AHR agonist and was injected twice daily, stocked in ethanol, and formulated in PEG-400 to a final dose of 50 ug/100 ul. Spleen and colon were collected at 6 days post vaccination; data is from 2 independent experiments. FIG. 3H (left panel) shows the percentage of OT-I cells among CD8a+ cells. The right panel shows the total OT-I T cell numbers recovered. Combination of AM80 and 10-CL-BBQ significantly increased the number of transferred OT-I cells in the colon (unpaired t-test with Welch's correction, comparing AM80+10-CL-BBQ to each other group).

Parenteral retinoic acid receptor agonist targets CD8+ T cell responses to the small intestine. FIG. 3I shows the quantification of flow cytometry analysis of the spleen, small intestine IEL, and small intestine LPL on day 7 after treatment with an RAR agonist. FIG. 3J shows quantification of cells/0.05 mm³ on day 7 after treatment with an RAR agonist.

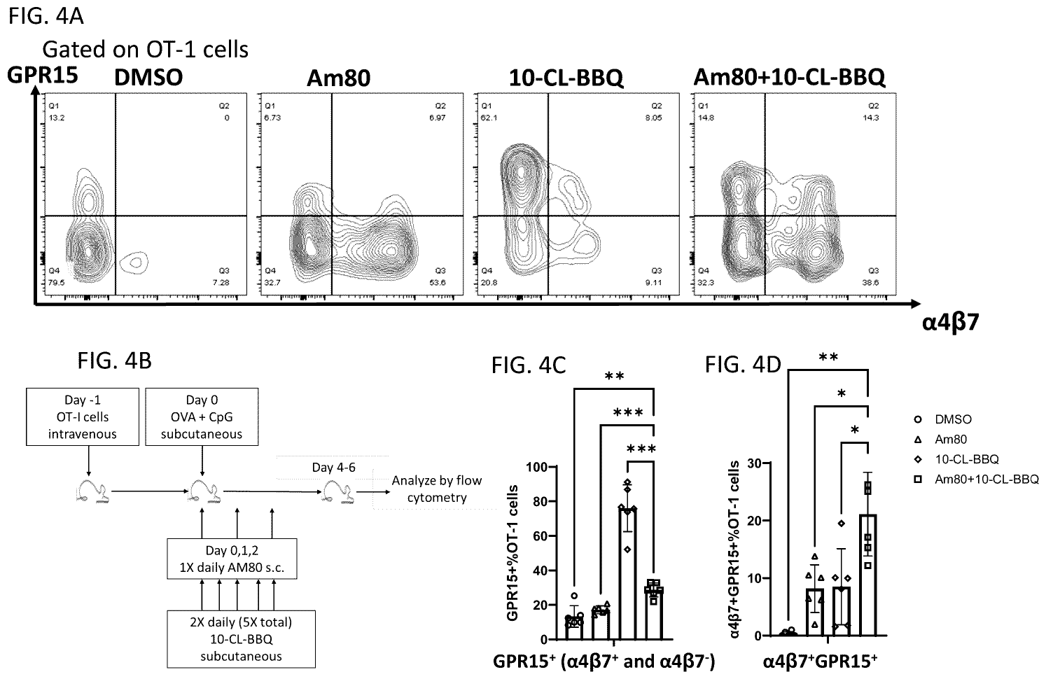

10-CL-BBQ induced GRP15 expression on effector T cells, while Am80+10-CL-BBQ induced α4β7+GPR15+ cells more robustly. Using the same vaccination regimen described in FIGS. 2 and 4B, blood was analyzed at timepoints between day 4-6 by flow cytometry. FIG. 4A shows a representative flow cytometry plots showing expression of α4β7 and GPR15 on OT-1 cells in the blood. Plots are pre-gated on OT-1 cells. As shown, 10-CL-BBQ alone induced the most robust GPR15 expression, while Am80+10-CL-BBQ induced the most robust α4β7+GPR15+ cells. FIG. 4C shows the percentage of GPR15+ cells out of total OT-1 cells. FIG. 4D shows the percentage of α4β7+GPR15+ cells out of total OT-1 cells. Both Brown-Forsythe and Welch's ANOVA with Dunnett's T3 multiple comparisons test were used to determine the p value. *p<0.05, **p<0.01, ***p<0.001, and ****p<0.0001. ns, not significant.

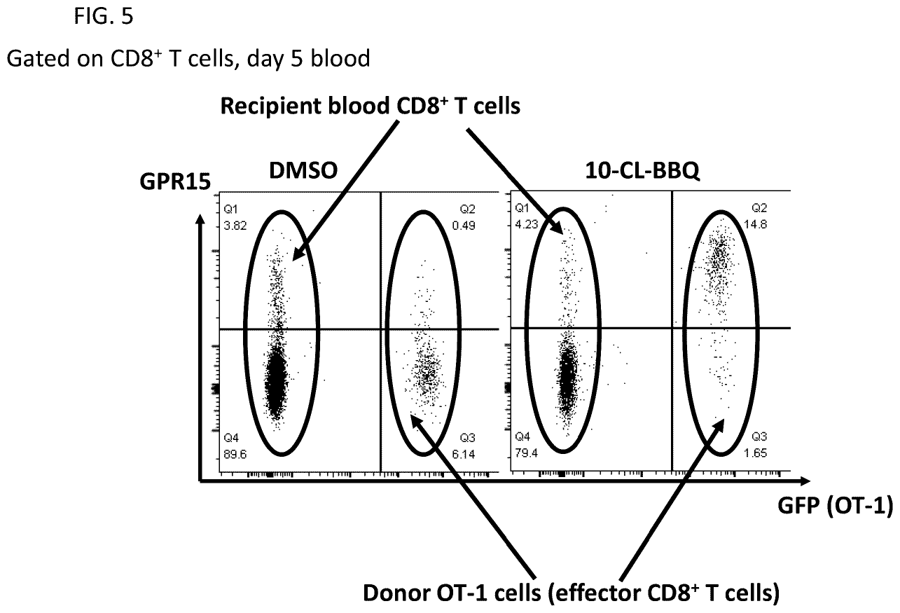

10-CL-BBQ up-regulated GRP15 expression on effector CD8+ T cells, but not in circulating non-stimulated CD8+ T cells. Using the same vaccination regimen described in FIG. 2, blood was analyzed at day 5 by flow cytometry. Representative flow cytometry plots showing expression of GPR15 on adoptively transferred OT-1 cells (GFP+) that's in the effector phase, and CD8+ T cells from the recipient animal (GFP−) that's mostly naïve. Plots are pre-gated on CD8a+ cells. As shown, 10-CL-BBQ mostly up-regulated GPR15 on OT-1 cells but did not affect GPR15 expression on CD8a+ cells in the blood of the recipient. (FIG. 5)

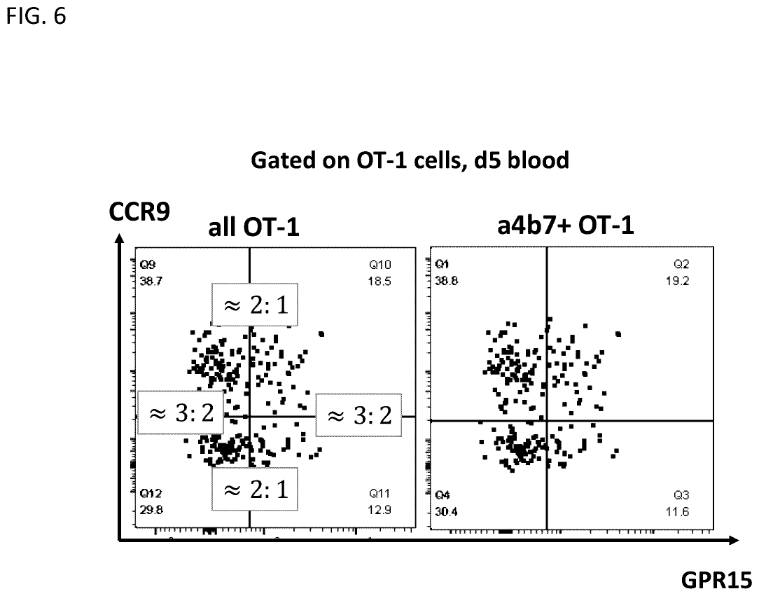

Using the same vaccination regimen described in FIG. 2, blood was analyzed at day 5 by flow cytometry. Representative flow cytometry plots of the AM80+10CLBBQ group showing expression of CCR9 and GPR15 on adoptively transferred OT-1 cells (GFP+), and in OT-1 cells that are α4β7+. Expression of CCR9 and GPR15 does not appear to be interacting with each other. (FIG. 6)

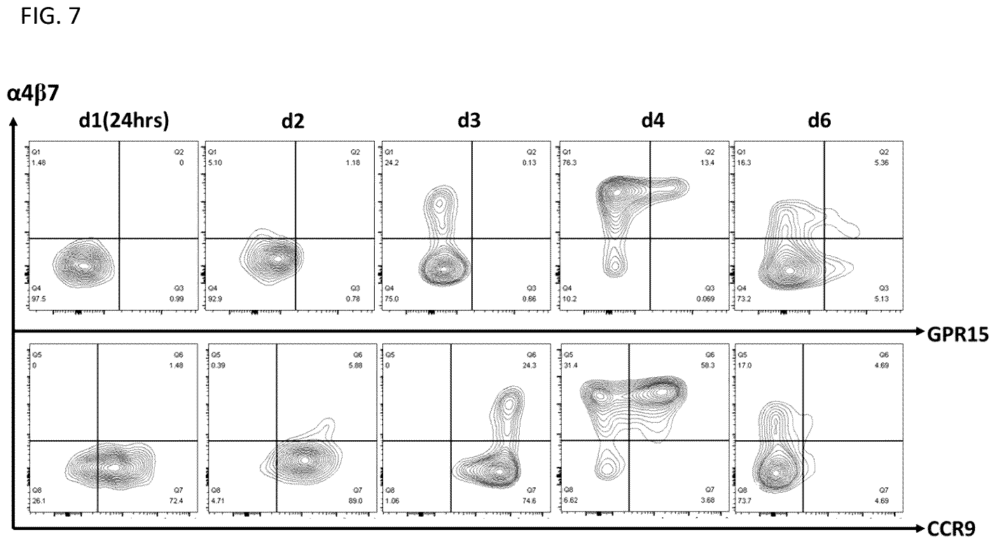

Expression of α4β7, CCR9 and GPR15 on CD8+ T cells after antigen encounter was temporally regulated, as shown in FIG. 7. Using the same adoptive transfer, immunization and treatment regimen, draining lymph nodes (brachial LNs) were harvested at different days to analyze the expression of gut homing molecules. Representative flow cytometry plots (pre-gated on OT-1 cells) from brachial lymph nodes of Am80+10-CL-BBQ treated mice. While CCR9 was induced within 24 hrs, the upregulation of GPR15 and generation of α4β7+GPR15+ cells started at day 4 (96 hours) in the LNs. Upregulation of GPR15 happened at the peak of α4β7 expression, and concurrently with the decline of CCR9 expression.

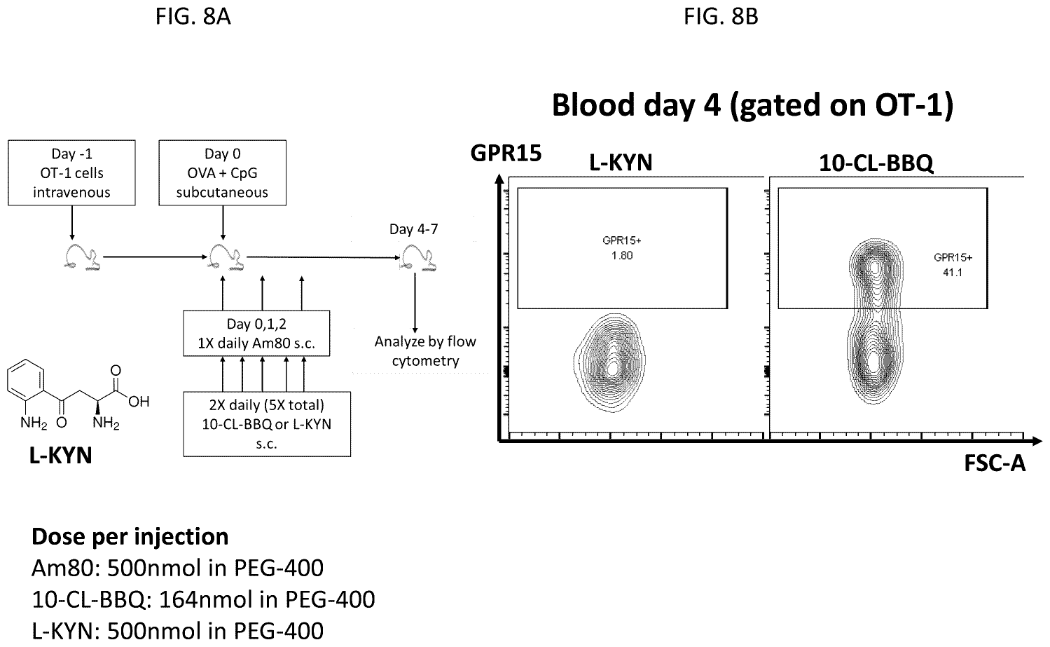

AHR ligand L-kynurenine (L-KYN), using the described dosing regimen, did not increase expression of GPR15 on CD8+ T cells, as shown in FIGS. 8A-8B. L-kynurenine (L-KYN), an endogenous AHR ligand, was tested for the ability to up-regulate GPR15 and promote large intestine trafficking in the parenteral vaccination model. L-KYN, at the disclosed experimental dose (500 nmol in PEG-400) and regimen, did not increase expression of GPR15 on effector CD8+ T cells. A representative flow cytometry plot of day 4 blood, pre-gated on OT-1 cells (FIG. 8B). While 10-CL-BBQ significantly up-regulated GPR15 on OT-1 cells, L-KYN did not have any effect.

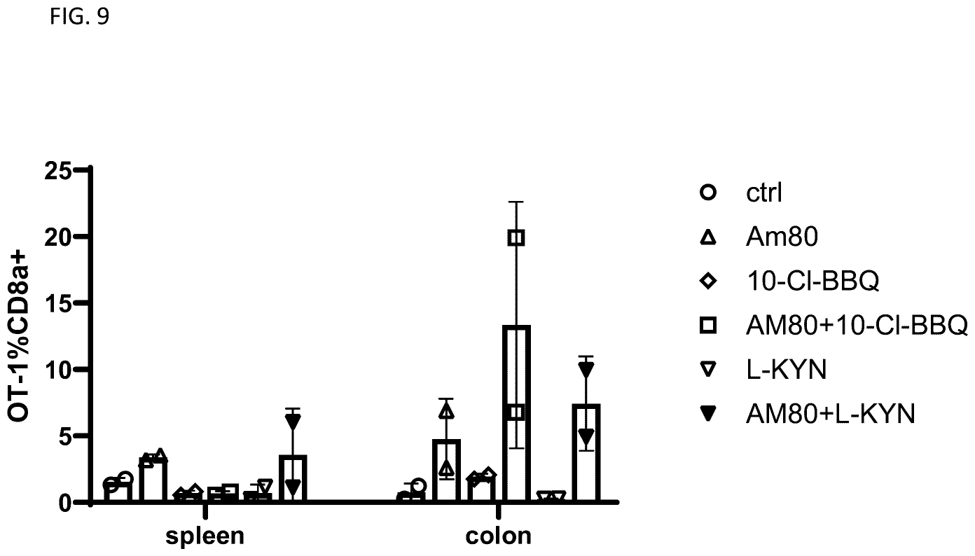

AHR ligand L-kynurenine (L-KYN), using the described dosing regimen, did not increase colon trafficking alone or in combination with Am80. FIG. 9 shows the percentage of OT-1 cells out of total CD8+ T cells, in the spleen and colon following vaccination and respective treatments.

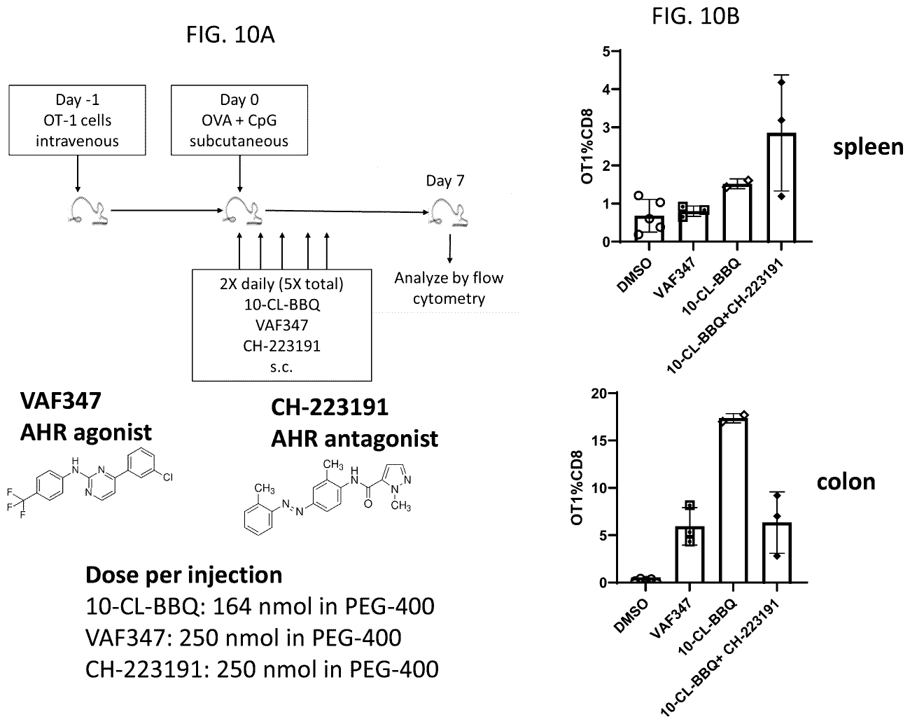

AHR agonist VAF347 increased colon trafficking at a lower efficacy than 10-CL-BBQ. Additionally, inhibition of AHR with CH-223191 antagonized the effects of 10-CL-BBQ on colon trafficking. AhR Agonist V, VAF347, a synthetic AHR agonist, was tested for the ability to promote large intestine trafficking in the parenteral vaccination model. VAF347 at an experimental dose (250 nmol in PEG-400) and regimen, increased colon trafficking but at a lower efficacy than 10-CL-BBQ. Additionally, injection of CH-223191, an AHR antagonist (250 nmol in PEG-400), antagonized the effects of 10-CL-BBQ on colon trafficking. FIG. 10B shows the percentage of OT-1 cells out of total CD8+ T cells, in the spleen and colon following vaccination and respective treatments.

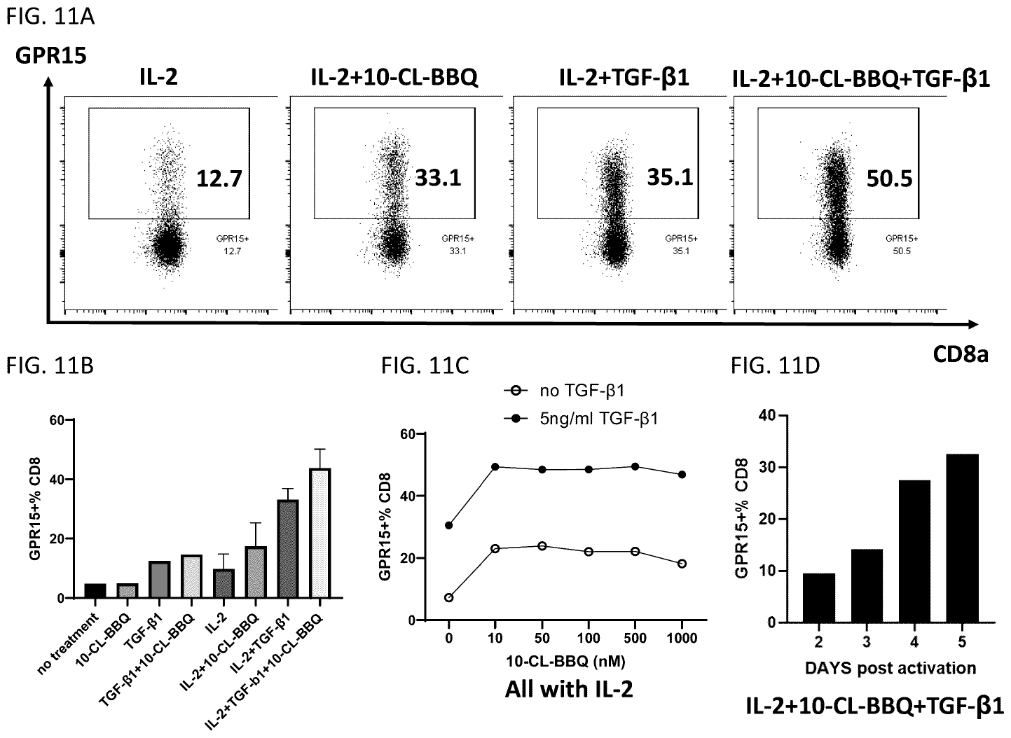

An in vitro method to generate GPR15+CD8+ T cells was established, as shown in FIGS. 11A-11D. Various treatment conditions were tested for the efficiency of generating GPR15⁺CD8⁺ T cells. OT-1 splenocytes were pulsed for 1 hour with 1 μg OVA peptide (257-264) and cultured overnight in complete RPMI media. The next day, CD8⁺ T cells were isolated using mouse CD8a⁺ T Cell Isolation Kit (Miltenyi) and cultured with various treatment conditions. In summary, combination of mouse IL-2 (5 ng/ml), TGF-β1 (5 ng/ml) and 10 nM 10-CL-BBQ yielded the most robust results. ***Particularly with the in vitro culture, there were some fluctuations between experiments on how much GPR15 was expressed, but overall conclusion remained the same. FIG. 11A shows an example flow cytometry plots of GPR15 expression in various treatment conditions. 4 days post activation. Plots were pre-gated on CD8a⁺ cells. FIG. 11B shows the percentage of GPR15⁺ cells out of total CD8⁺ T cells, 4-5 days post activation. FIG. 11C shows the effect of different doses of 10-CL-BBQ, with IL-2, with or without TGF-β1,4-5 days post activation. FIG. 11D shows an experiment showing percentage of GPR15⁺ cells out of total CD8⁺ T cells over time. GPR15⁺ T cells peaked around 4-5 days post activation. Cells were treated with IL-2+10-CL-BBQ+TGF-β1.

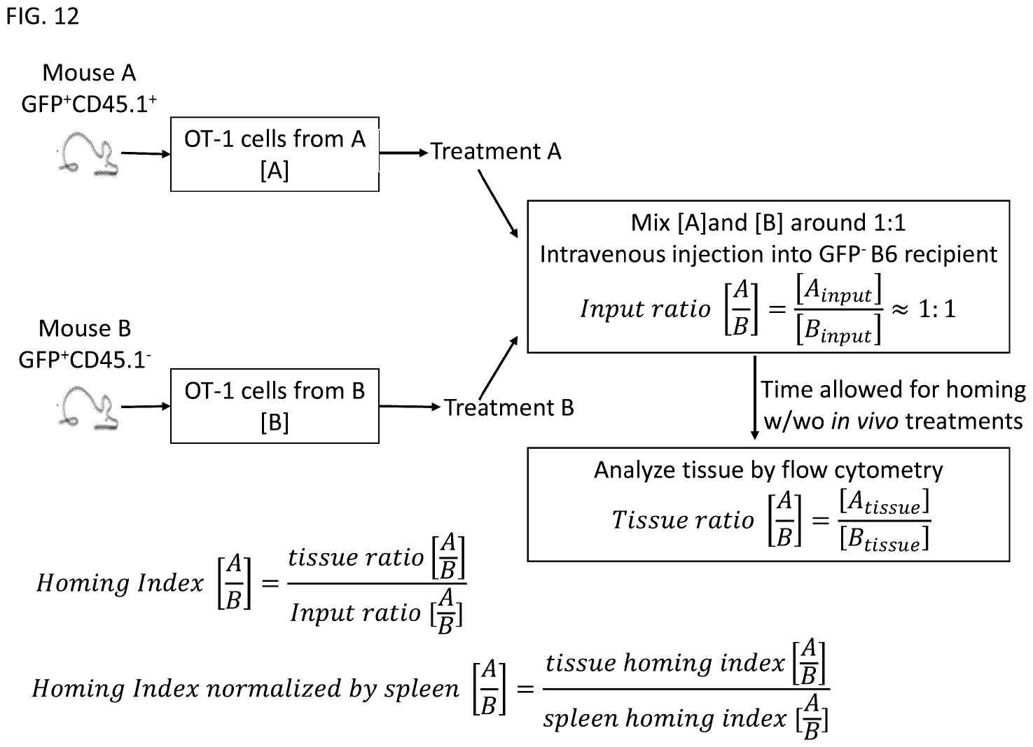

A schematic of homing and competitive homing assays using OT-1 cells and associated calculations is shown in FIG. 12. Briefly, two GFP+OT-1 transgenic mice with or without expression of the congenic marker CD45.1 were used per experiment (mouse A and mouse B), and OT-1 cells were isolated from their spleens. OT-1 cells from different mice ([A] and [B]) were subjected to different treatments (A and B, including culture, genetic manipulation, in vivo activation and re-harvesting, etc). The cells were then mixed at an around 1:1 ratio and injected into a B6 recipient (GFP−). At timepoints (determined for each experiment), with or without additional in vivo treatments (determined by each experiment), tissues were analyzed by flow cytometry and the ratio of [A] and [B] in each tissue was obtained. Homing index and normalized homing index (see equation) were calculated. A high homing index [A/B](>1) means [A] is over-represented in the analyzed tissue compared to [B], suggesting that the treatments that [A] underwent made them better at trafficking to and staying in the analyzed tissue.

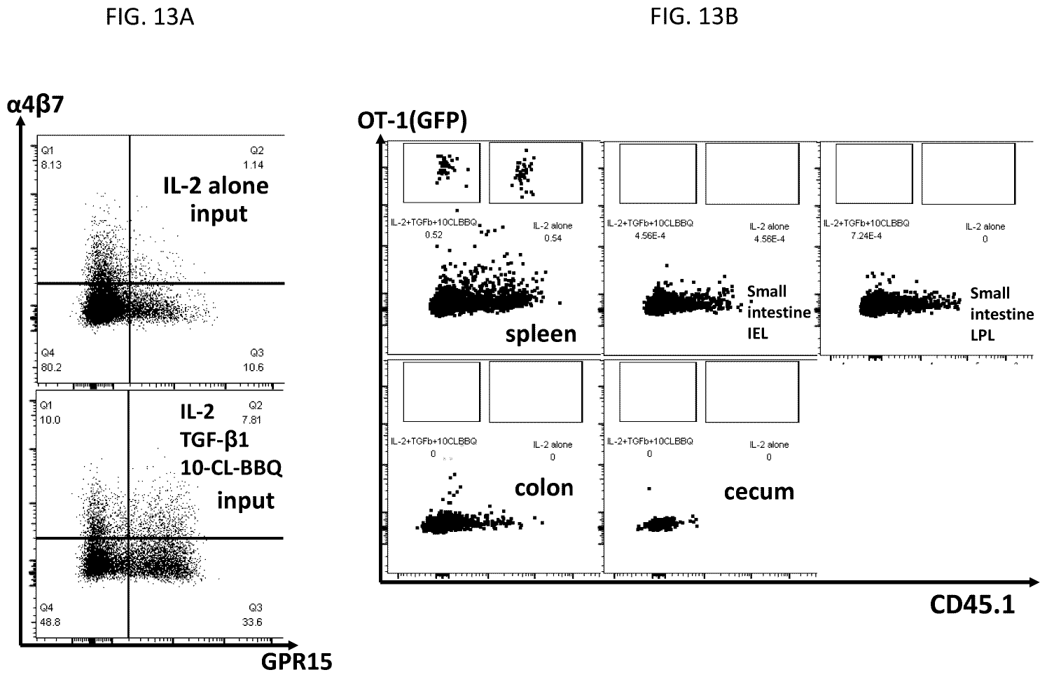

Targeting with 10-CL-BBQ alone, without Am80, did not constitute trafficking to small and large intestine. Cells were either cultured in vitro with IL-2 alone, or with the “optimized” method for GPR15 expression (IL-2+10-CL-BBQ+TGF-β1). The two culture conditions were distinguished by expression of congenic markers 45.1 and mixed cells (5×10⁶ cells from each treatment) were transferred into naïve B6 recipients. While the previously described method generated GPR15+ cells robustly compared to IL-2 alone, neither condition generated cells capable of trafficking to the gut, likely due to low levels of α4β7 expression. FIG. 13A shows expression of α4β7 and GPR15 in input cells (cells treated with IL-2 alone were 45.1+, and cells treated with the “optimized” method was 45.1−). FIG. 13B shows a representative plots of the spleen, small intestine (IEL and LPL), colon and cecum. Transferred cells were gated on GFP and CD45.1. While both populations were detected in the spleen, none of the transferred cells could be detected across all intestinal compartments.

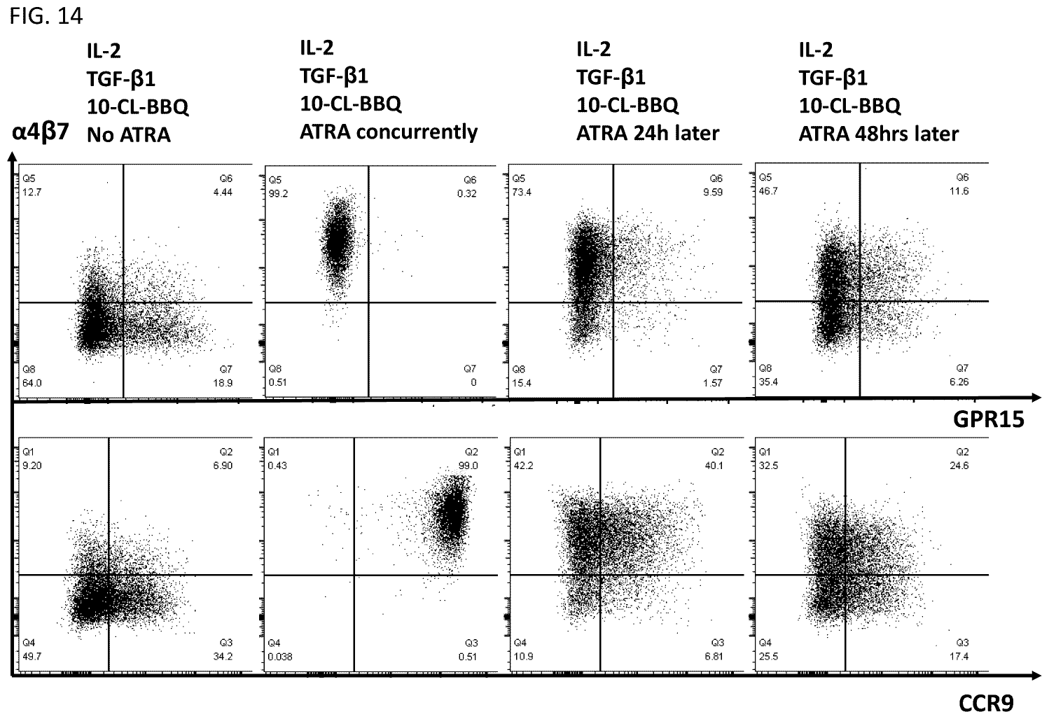

An in vitro method to generate α4β7^{high} GPR15+CD8+ T cells was established, as shown in FIG. 14. 10 nM all-trans retinoic acid (ATRA) was added to the cell culture at different time points, and characterized the expression of α4β7, GPR15 and CCR9 at day 4 post activation. While adding ATRA concurrently with IL-2+ TGF-β 1+10-CL-BBQ completely abrogated GPR15 expression and skewed cells towards a small intestine trafficking phenotype (α4β7^{high}CCR9+), adding ATRA 24-48 hours after IL-2+ TGF-β1+10-CL-BBQ enabled the generation of an α4β7^{high} GPR15+ population.

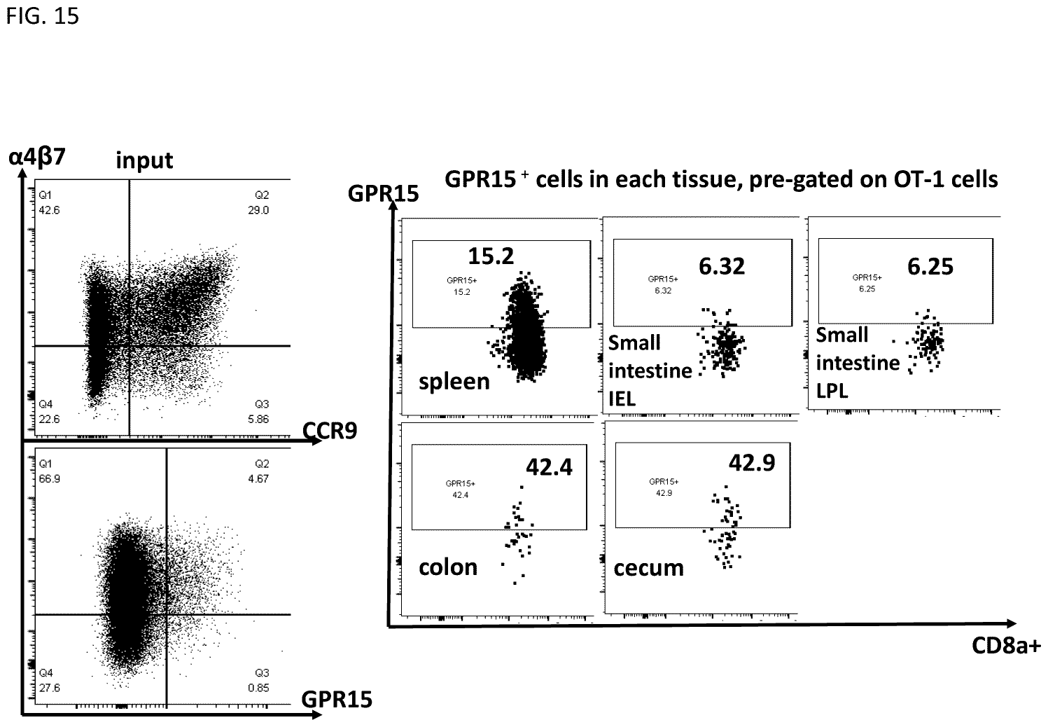

α4β7^{high}GPR15+ cells were generated using the method described in FIG. 14, and transferred approximately 1×10⁷ cells into a naïve B6 recipient. In the spleen, small intestine (IEL and LPL), colon and cecum, transferred cells (plots pre-gated on OT-1 cells) were identified and gated on (FIG. 15). Furthermore, OT-1 cells recovered from the colon and cecum preferentially showed increased GPR15 expression. This lends additional support to GPR15 playing a role in CD8+ T cell trafficking to the large intestine.

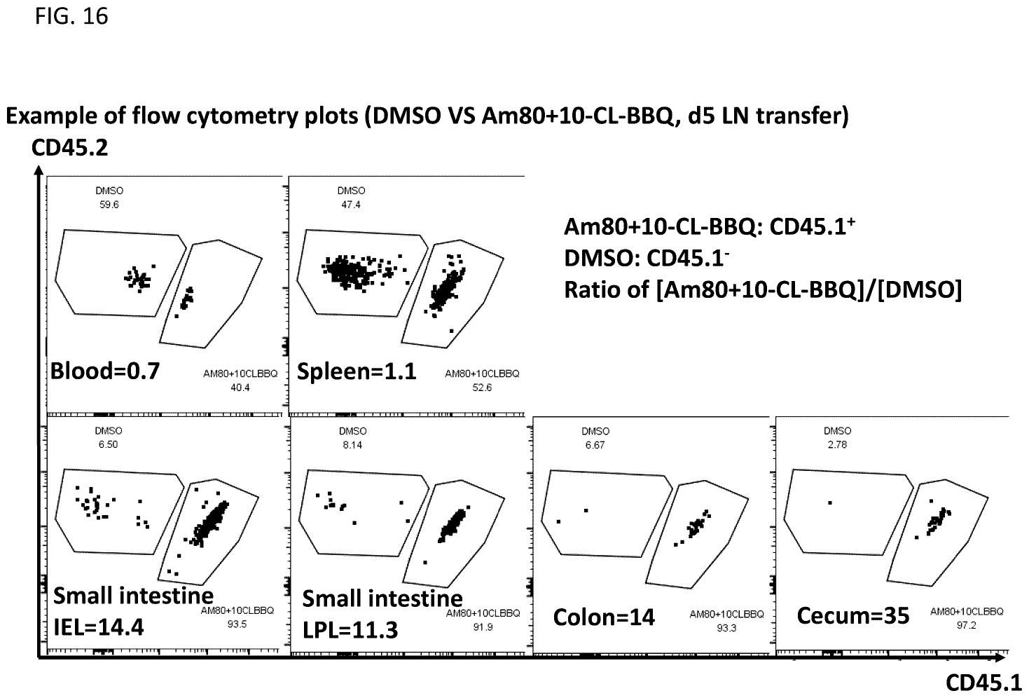

As a supplementation to in vitro approaches, the gut homing capacity of in vivo generated effector T cells was tested in the disclosed vaccination strategy. Following vaccination and treatment with DMSO, Am80, 10-CL-BBQ or Am80+10-CL-BBQ, draining lymph nodes (brachial LNs) were harvested at day 4-5. Effector OT-1 cells from different treatment groups were mixed in competitive homing experiments and distinguished by expression of CD45.1. 10\hat{ }5 OT-1 cells from each treatment group were transferred into naïve B6 recipients, and tissues were harvest one day later for flow cytometry analysis. T_{eff} cells generated in vivo with Am80+10-CL-BBQ increased trafficking to the colon and cecum compared to DMSO, Am80 alone or 10-CL-BBQ alone, as shown in FIG. 16. Flow cytometry plots from a competitive homing experiment compared Am80+10-CL-BBQ versus DMSO. OT-1 cells from the Am80+10-CL-BBQ treated animals were significantly over-represented in all intestinal tissues compared to cell ratio in the spleen and blood.

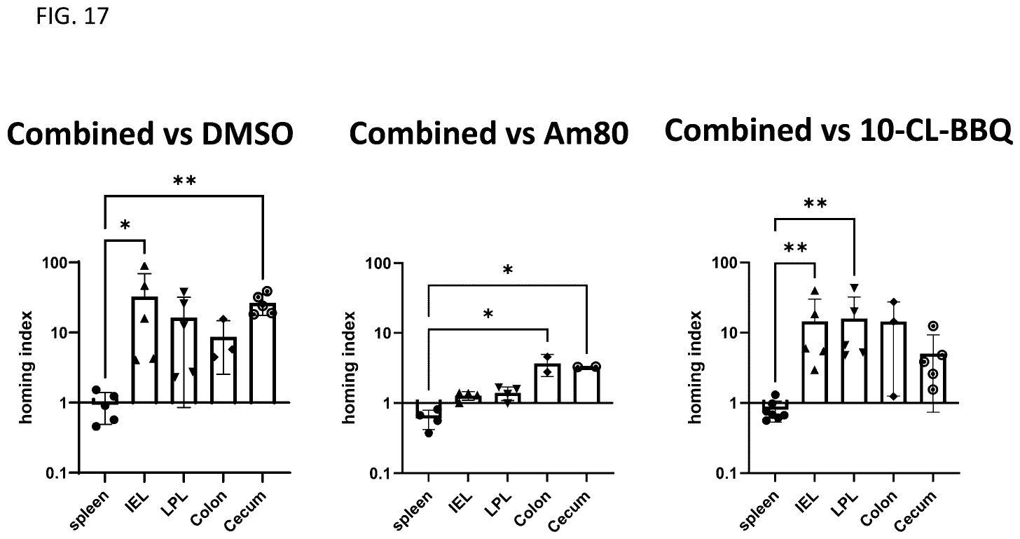

T_{eff} cells generated in vivo with Am80+10-CL-BBQ increased trafficking to the colon and cecum compared to DMSO, Am80 alone or 10-CL-BBQ alone. Summary plots of in vivo competitive homing are shown in FIG. 17. OT-1 cells from combined treatment group (Am80+10-CL-BBQ) showed preferential trafficking to the colon and cecum when compared to any other treatment condition (DMSO, Am80 alone, 10-CL-BBQ alone). Combined treatment group also showed preferential trafficking to the small intestine (IEL and LPL) when compared to DMSO and 10-CL-BBQ groups, but not compared to Am80 group. Normalized to input ratio. Kruskal-Wallis test and Dunn's multiple comparisons test. *p<0.05, **p<0.01, ***p<0.001, and ****p<0.0001. ns, not significant.

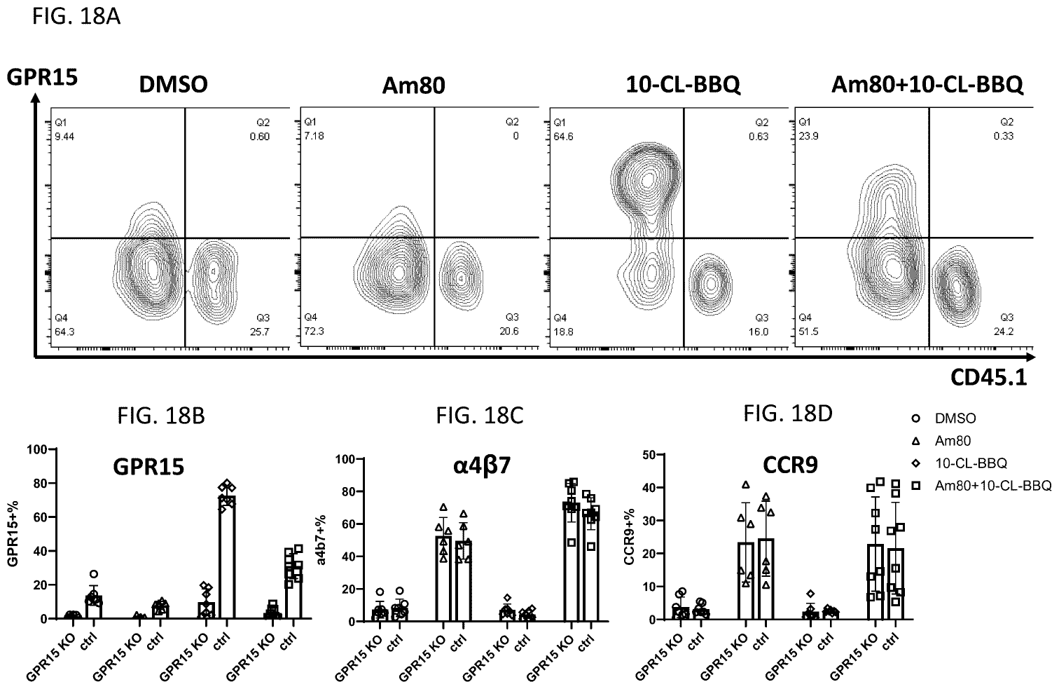

Lack of GPR15 does not affect α4β7 and CCR9 expression. Loss of GPR15 gene by CRISPR knockout abrogated GPR15 expression but did not affect expression of α4β7 and CCR9. Naïve GFP+OT-1 cells were nucleofected with Cas9 protein, complexed with either control (CD19) gRNA, or GPR15 gRNA. Transfected cells were distinguished based on expression of congenic marker CD45.1 and co-transferred into recipient mice. Recipient mice were immunized with OVA+CpG, and injected with DMSO, Am80, 10-CL-BBQ or Am80+10-CL-BBQ. FIG. 18A depicts representative flow cytometry plots, gated on OT-1 cells. At day 5 in the blood, OT-1 cells nucleofected with GPR15 gRNA (CD45.1+) almost fully abrogated GPR15 expression, compared to OT-1 cells nucleofected with control gRNA (CD45.1−). Quantification of GPR15+, α4β7+ and CCR9+ cells, percentage out of OT-1 cells in respective groups, are shown in FIGS. 18B, 18C, and 18D, respectively. Control cells used were CD45.1− and GPR15 knockout cells were CD45.1+.

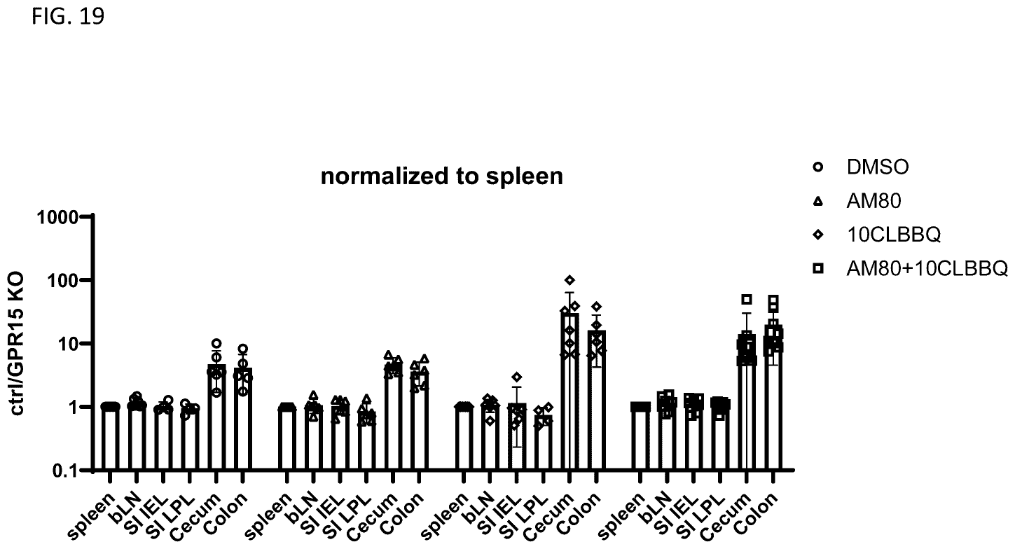

GPR15 deficiency impaired trafficking to the large intestine, as shown in FIG. 19. Decreased trafficking of OT-1 cells to spleen, bLN, SI IEL, SI LPL, cecum, and colon in mice 7 days after immunization was seen. Ratio of [Ctrl]:[GPR15 knockout] OT-1 cells in tissues at 7 days post immunization and normalized to spleen was calculated for each condition tested.

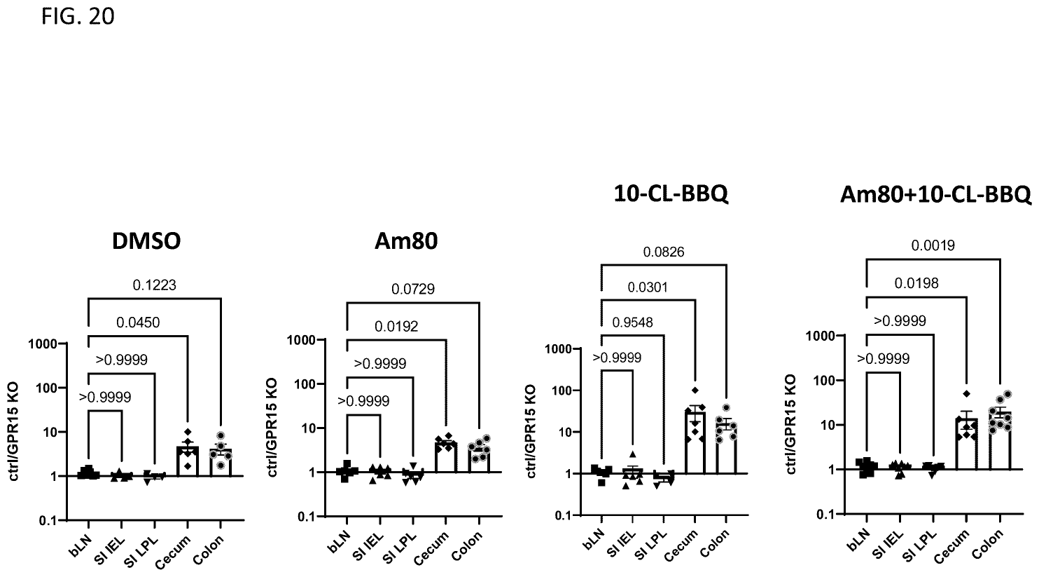

The level of trafficking of OT-1 cells to bLN, SI IEL, SI LPL, cecum, and colon in mice 7 days after immunization and treatment with either DMSO, Am80, 10-CL-BBQ, or Am80+CL-10-BBQ are shown in FIG. 20. Consistent with the level of GPR15 expression, at day 7, GPR15 KO cells were under-represented in the colon and cecum in all groups. Ratio of [Ctrl]:[GPR15 knockout] and normalized to spleen. Kruskal-Wallis test and Dunn's multiple comparisons test. *p<0.05, **p<0.01, ***p<0.001, and ****p<0.0001. ns, not significant.

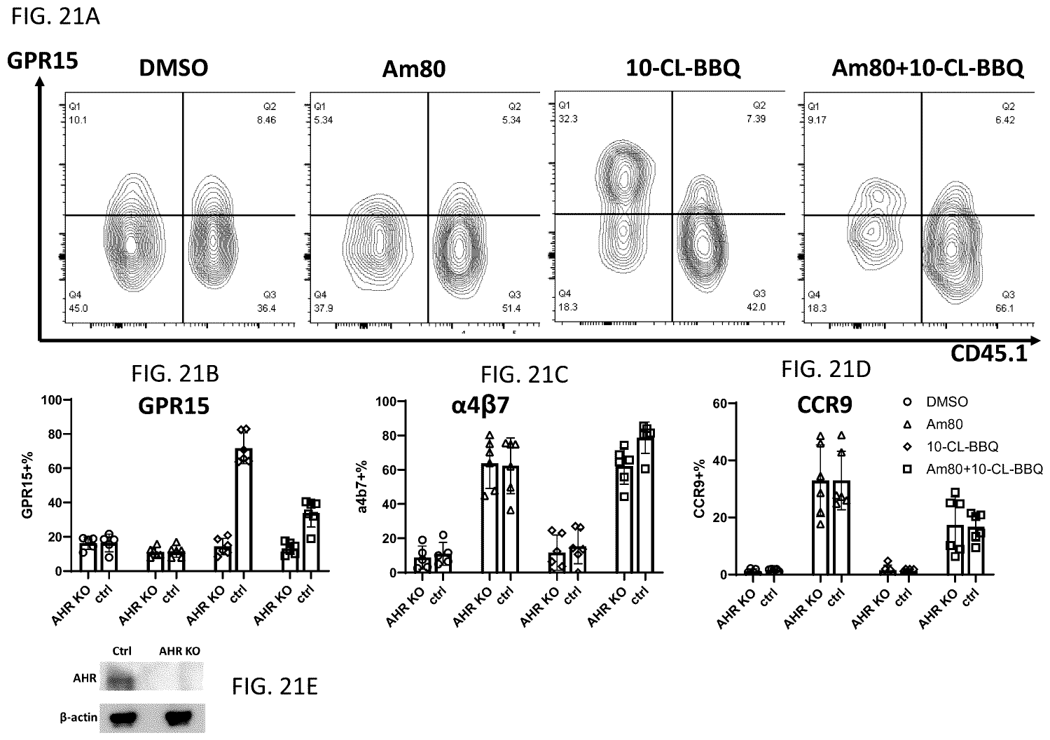

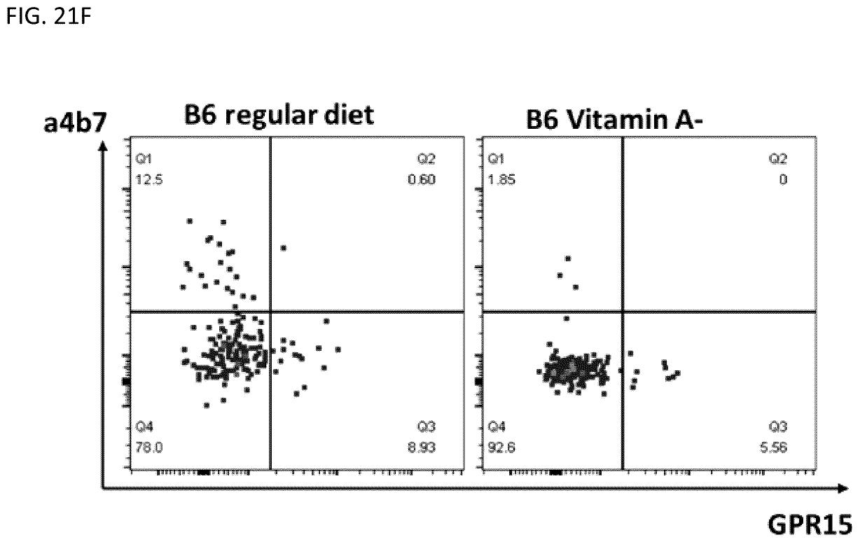

Loss of AHR abrogated ligand-depended upregulation of GPR15. Naïve GFP+OT-1 cells were nucleofected with Cas9 protein, complexed with either control (CD19) gRNA, or AHR gRNA. Transfected cells were distinguished based on expression of congenic marker CD45.1 and co-transferred into recipient mice. Recipient mice were immunized with OVA+CpG, and injected with DMSO, Am80, 10-CL-BBQ or Am80+10-CL-BBQ (same dose and regimen as previously described). Flow cytometry plots, gated on OT-1 cells. At day 5 in the blood, OT-1 cells nucleofected with AHR gRNA (CD45.1+) showed baseline expression of GPR15, but not 10-CL-BBQ dependent upregulation of GPR15, compared to OT-1 cells nucleofected with control gRNA (CD45.1−). Quantification is shown of GPR15+, α4β7+ and CCR9+ cells, percentage out of OT-1 cells in respective groups (FIGS. 21B-21D). FIG. 21E shows a Western blot confirming successful knockout of AHR, with β-actin as control. Control cells used were CD45.1⁻ and GPR15 knockout cells were CD45.1+.

FIG. 21F shows expression of α4β7 and GPR15 when mice are feed a regular diet or a Vitamin A deficient diet. Vitamin A deficiency decreased expression of α4β7. However, GPR15 expression was similar to the control.

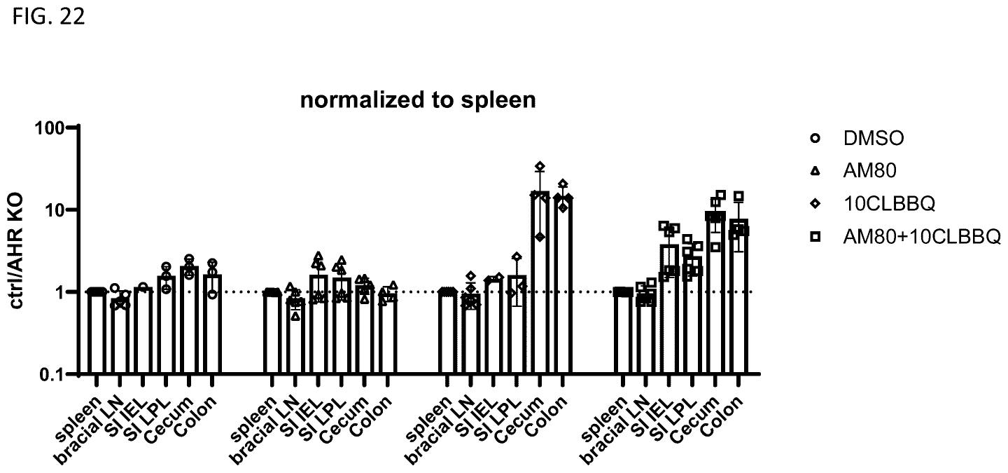

AHR deficiency impaired ligand-dependent colon and cecum trafficking. The ratio of [Ctrl] to [AHR knockout] OT-1 cells in tissues at 7 days post immunization, normalized to spleen are shown in FIG. 22. Consistent with the level of GPR15, at day 7, AHR KO cells were under-represented in the colon and cecum in the 10-CL-BBQ and Am80+10-CL-BBQ groups, but not in the DMSO and Am80 groups.

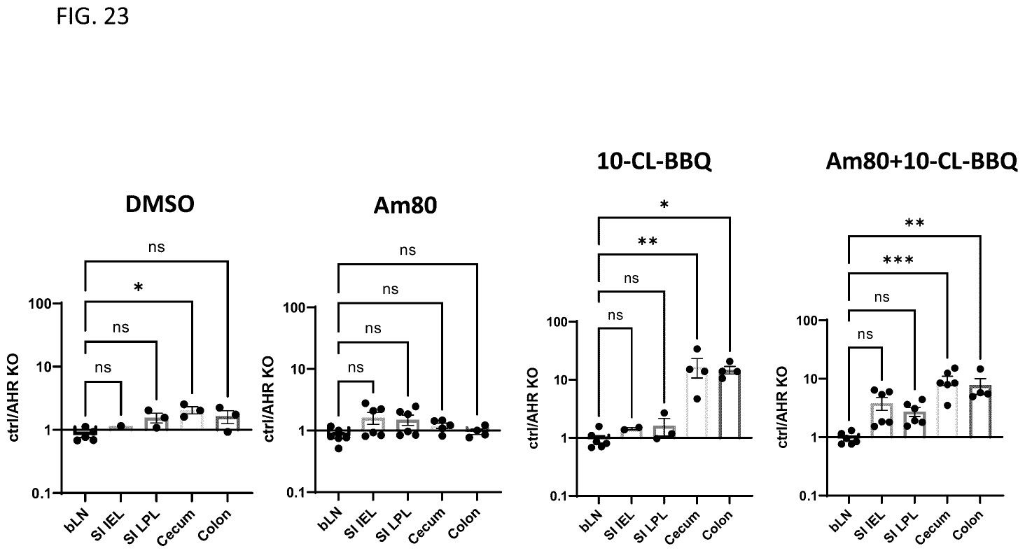

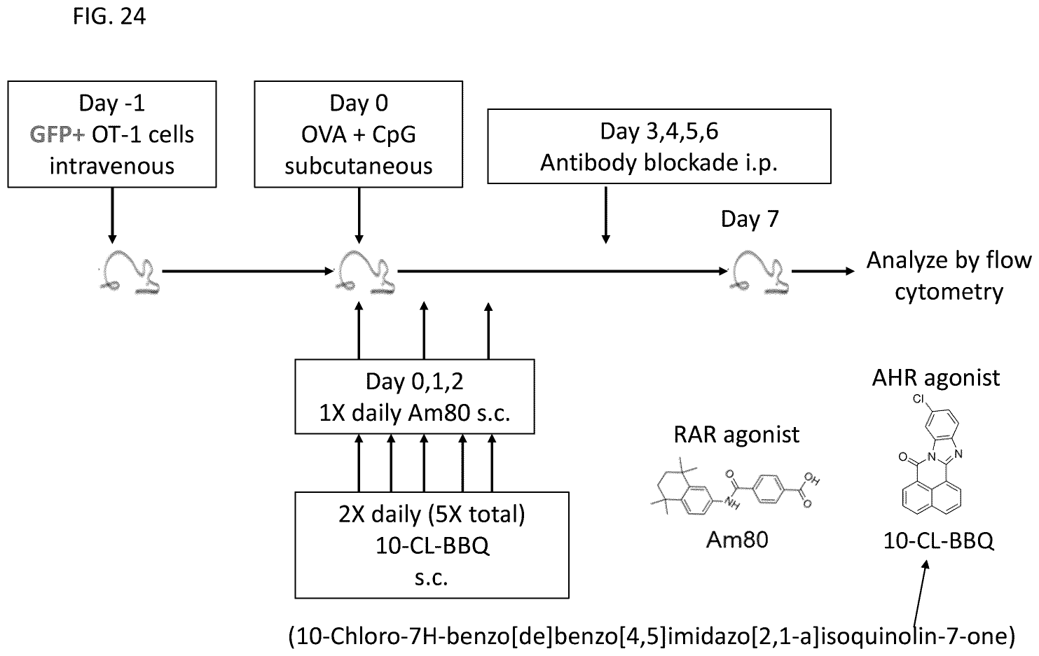

FIG. 23 shows the ratio of [Ctrl] to [AHR knockout] OT-1 cells in tissues at 7 days post immunization, normalized to spleen. the combination of Am80+10-CL-BBQ showed an increased on OT-1 cells moving to the gut, especially the colon. Kruskal-Wallis test (one-way ANOVA) and Dunn's multiple comparisons test (pair-wise with bLN as ctrl). *p<0.05, **p<0.01, ***p<0.001, and ****p<0.0001. ns, not significant.

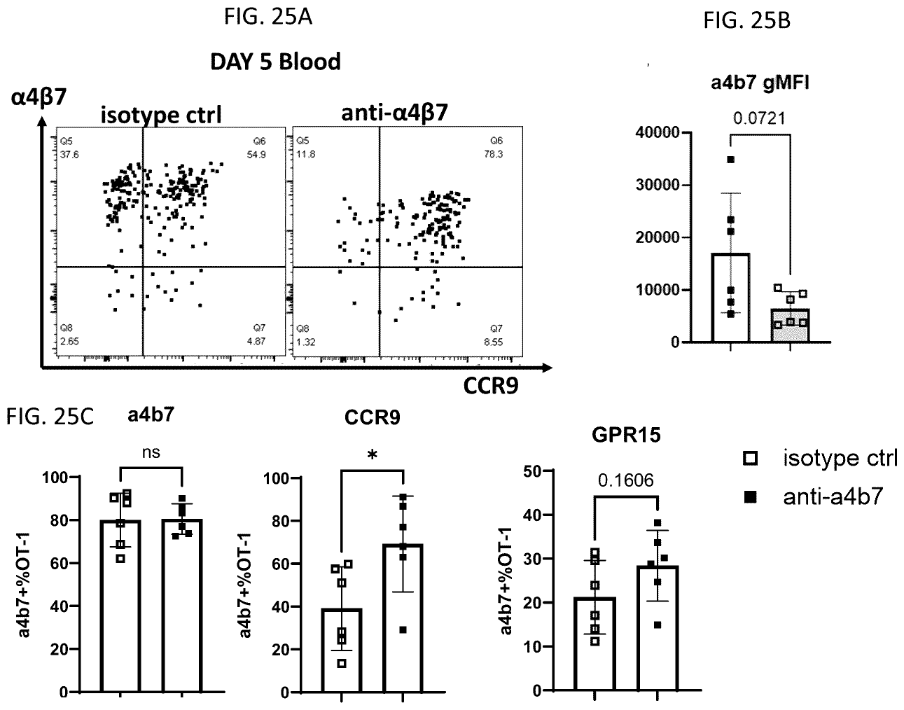

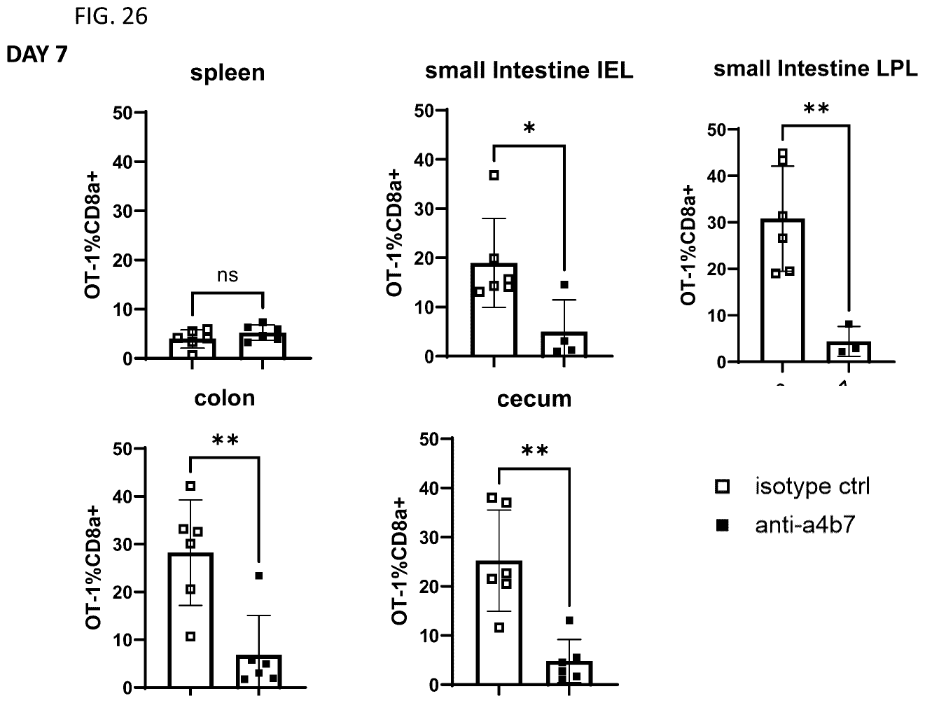

Antibody blockade of α4β7 was used to determine the effect on expression of homing molecules in the blood. OT-1 cells were transferred into B6 mice and recipients were immunized s.c. with OVA+CpG on day 0, and injected with Am80+10-CL-BBQ on day 0, 1 and 2. Either anti-mouse α4β7 blocking antibody or isotype control antibody was given i.p. at 100 ug/100 ul per day on day 3,4,5 and 6. At day 5, blood was analyzed by flow cytometry. Blockade of α4β7 reduced signal intensity of α4β7 on OT-1 cells, but did not alter the percentage of α4β7+ cells. Blockade of α4β7 also altered the presence of CCR9+ cells in the blood, and presence of GPR15+ cells (not significant). Blood at day 5 was analyzed by flow cytometry and gating OT-1 cells. OT-1 cells in the anti-α4β7 treated mice showed lower staining of α4β7 (using the same antibody clone). Blood samples at day 5 were analyzed for expression levels of gMFI (geometric mean flow cytometry) of α4β7 on OT-1 cells. FIG. 25C shows the percentage of OT-1 cells that expressed α4β7, CCR9 and GPR15 in day 5 blood. Welch's t test. *p<0.05, **p<0.01, ***p<0.001, and ****p<0.0001. ns, not significant.

Antibody blockade of α4β7 impaired gut trafficking following parental vaccine with Am80+10-CL-BBQ. Antibody blockade of α4β7 reduced OT-1 T cells in the small and large intestine. OT-1 cells were transferred into B6 mice and recipients were immunized s.c. with OVA+CpG on day 0, and injected with Am80+10-CL-BBQ on day 0, 1 and 2. Either anti-mouse α4β7 blocking antibody or isotype control antibody was given i.p. at 100 ug/100 ul per day on day 3,4,5 and 6. At day 7, tissues were harvested and analyzed by flow cytometry. Blockade of α4β7 reduced OT-1 cells in the small and large intestine, without affecting the spleen. Percentage of OT-1 cells in respective tissues at day 7. Mice treated with α4β7 blocking antibody showed lower percentage of OT-1 cells in the small and large intestine. Welch's t test. *p<0.05, **p<0.01, ***p<0.001, and ****p<0.0001. ns, not significant.

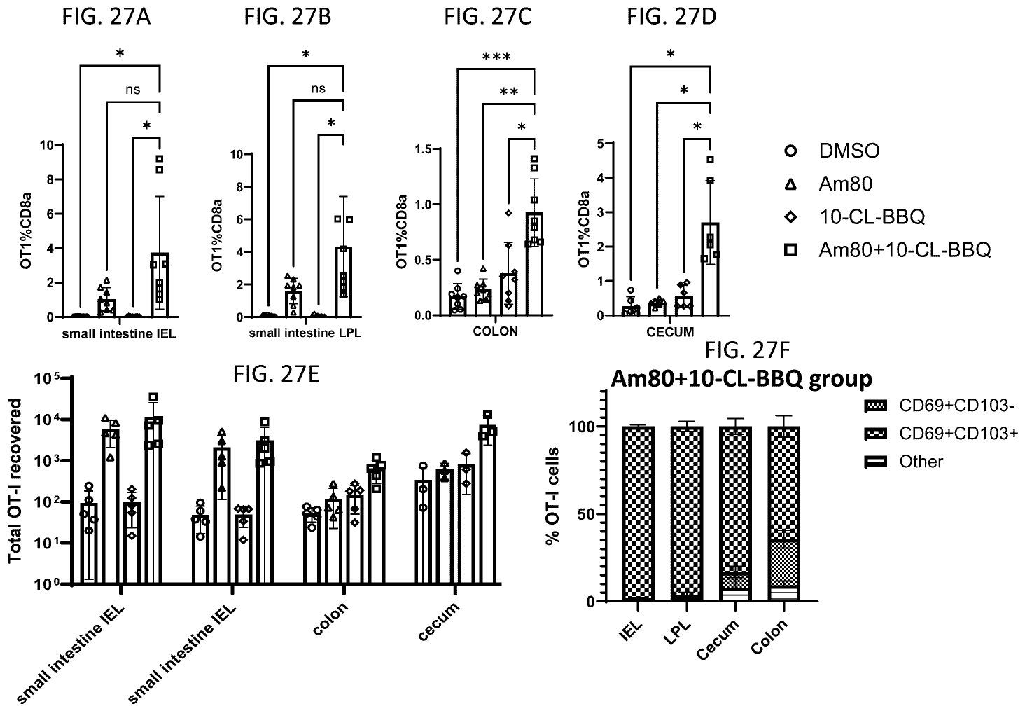

Am80+10-CL-BBQ increased establishment of resident memory cells in the gut. Characterization of resident memory cells in the small and large intestine are shown in FIGS. 27A-27F. Wildtype B6 recipients were adoptively transferred with 2×10⁴ GFP+OT-1 cells and immunized and treated with the same strategy as described previously. Spleen, small intestine (IEL and LPL), cecum and colon were harvested at 3-4 months and analyzed by flow cytometry.

Am80+10-CL-BBQ generated the highest percentage/number of resident memory OT-1 cells in all intestinal tissues, characterized by the expression of CD103 and CD69. Percentage of OT-1 cells out of CD8a+ cells in small intestine IEL, small intestine LPL, colon, and cecum are shown in FIGS. 27A-27D. The number of OT-1 cells recovered from the small intestine (IEL), small intestine (LPL), colon, and cecum show increased OT-1 cells when treated with the combination of Am80+10-CL-BBQ in each of the gut samples. FIG. 27G shows expression of CD69 and CD103 in intestinal tissue resident OT-1 cells generated by combined treatment of Am80+10-CL-BBQ. Brown-Forsythe and Welch's ANOVA with Dunnett's T3 multiple comparisons test. *p<0.05, **p<0.01, ***p<0.001, and ****p<0.0001. ns, not significant.

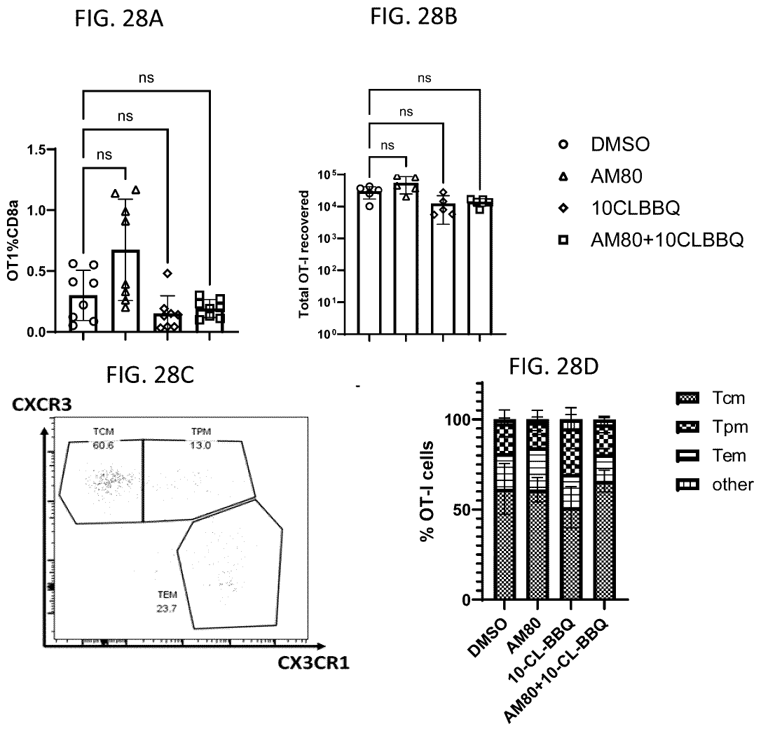

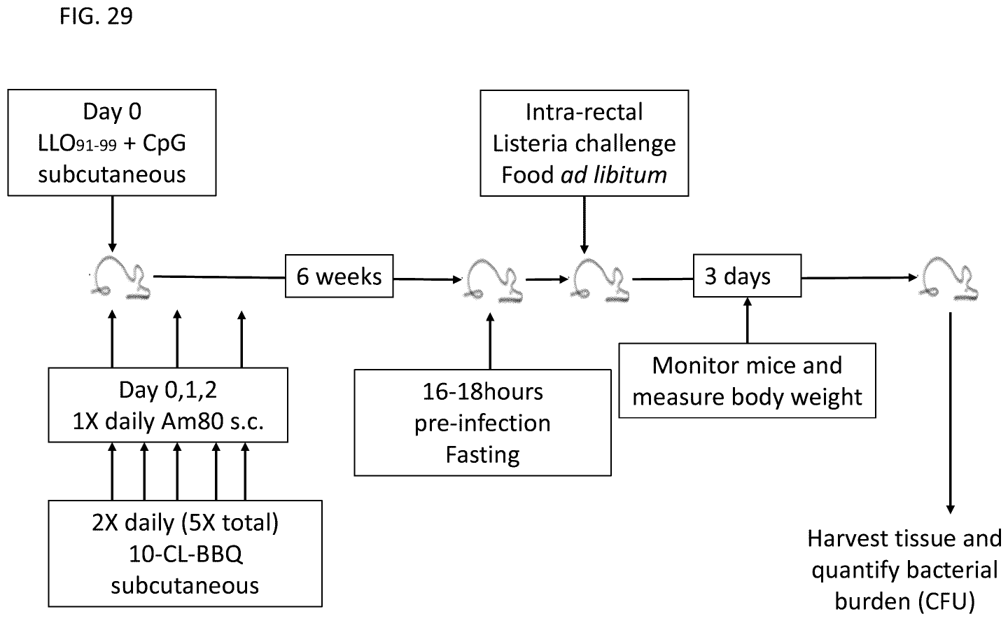

Am80+10-CL-BBQ did not impair generation and composition of systemic CD8+ T cell memory responses. Spleens were harvested and analyzed by flow cytometry. Am80+10-CL-BBQ did not impair generation and composition of systemic CD8+ T cell memory responses. FIG. 28A shows the percentage of OT-1 cells out of CD8a+ cells found in the spleen after treatment. FIG. 28B shows the number of total OT-1 cells recovered found in spleen after treatment. FIG. 28C shows a representative flow cytometry plot of circulating memory cells subsets (Tem-central memory, Tpm-peripheral memory, and Tem-effector memory cells) as defined by expression of CXCR3 and CX3CR1. Data was from spleen of an Am80+10-CL-BBQ treated mouse, gated on OT-1 cells. FIG. 28D shows the percentage of each circulating memory cell subset out to total OT-1 cells in the spleen. Brown-Forsythe and Welch's ANOVA with Dunnett's T3 multiple comparisons test. *p<0.05, **p<0.01, ***p<0.001, and ****p<0.0001. ns, not significant.

The intra-rectal Listeria infection model included the following: Female BALB/c mice were immunized subcutaneously with 50 ug LLO91-99 peptide+CpG-1668 on day 0, and injected with DMSO, Am80, 10-CL-BBQ or Am80+10-CL-BBQ on day 0, 1 and 2. Six weeks post vaccination, mice were fasted overnight, and the next day inoculated intra-rectally with 4×109 CFU Listeria. For the infection, mice were fasted overnight, anesthetized with ketamine+xylazine, and the inoculum was slowly delivered in 20 μl volume. Mice were monitored daily, and colon and spleen were harvested 3 days post infection, weighed and plated on BHI agar plates for CFU count.

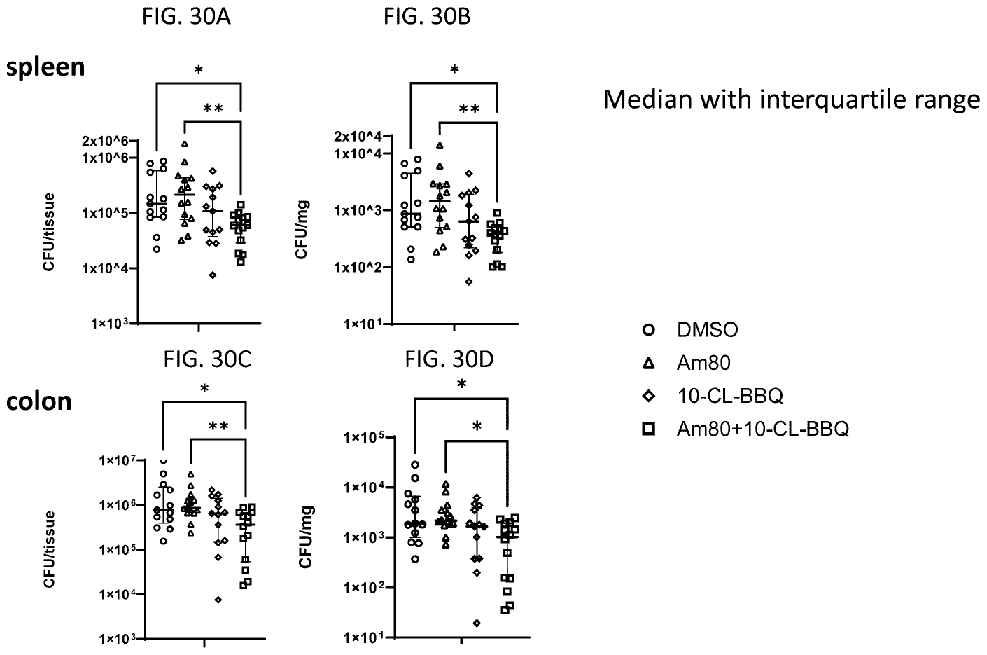

Am80+10-CL-BBQ improved vaccine protection against intra-rectal Listeria infection. Bacterial Burden following intra-rectal Listeria challenge was characterized. Compared to DMSO, Am80 and 10-CL-BBQ groups, mice treated with Am80+10-CL-BBQ showed lower bacterial burden in the colon and spleen, indicative of stronger vaccine protection. FIG. 30A shows colony forming units (CFU) in the spleen, per tissue and (FIG. 30B) per mg tissue. FIG. 30C shows CFU in the colon, per tissue and (FIG. 30D) per mg tissue; Kruskal-Wallis test with Dunn's multiple comparison. *p<0.05, **p<0.01, ***p<0.001, and ****p<0.0001. ns, not significant.

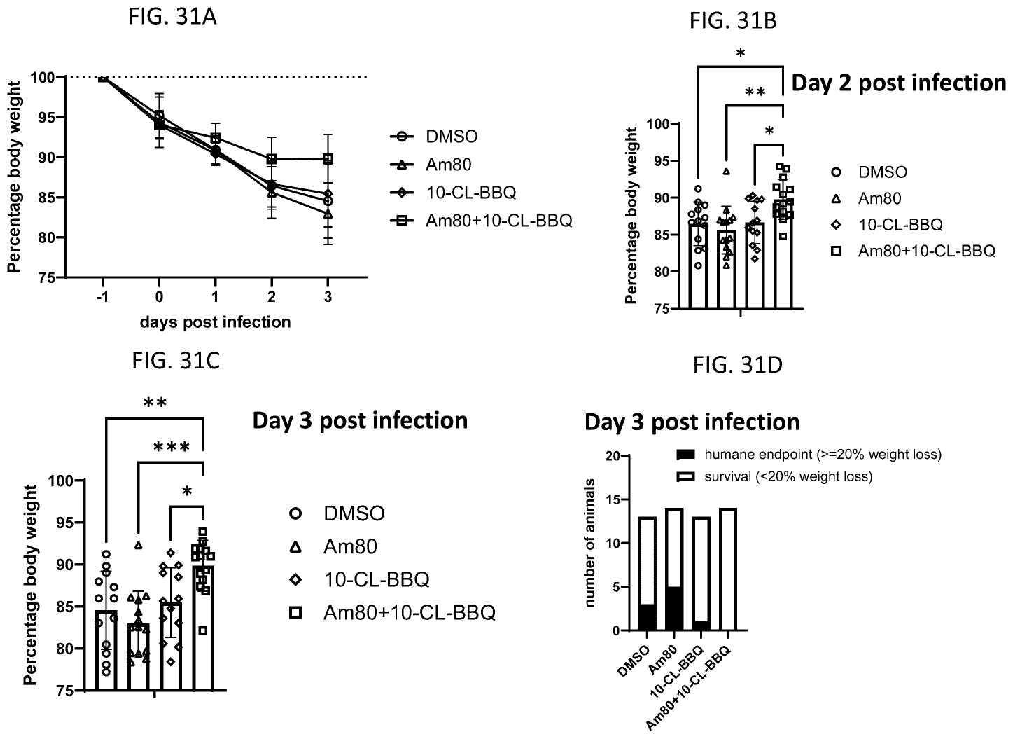

Weight loss following intra-rectal Listeria challenge was determined. Compared to DMSO, Am80 and 10-CL-BBQ groups, mice treated with Am80+10-CL-BBQ showed less weight loss, indicative of stronger protection against infection/disease symptoms. (FIG. 31A) Weight curve (percentage of starting weight); percentage body weight at day 2 (FIG. 31B) and day 3 (FIG. 31C) following infection; (FIG. 31D) number of mice reaching humane endpoint at day 3 (weight loss >=20%). Kruskal-Wallis test with Dunn's multiple comparison. *p<0.05, **p<0.01, ***p<0.001, and ****p<0.0001. ns, not significant.

### Example 3: Applications

While stimulation of RAR or AHR alone increased lymphocyte homing to the colon only to a small degree, the combined stimulation of RAR and AHR lead to significantly more effective colon imprinting. This synergistic effect is the result of upregulation of two indispensable traffic molecules, the α4β7 integrin, which is induced by RAR stimulation via ATRA or Am80, and a G-protein coupled receptor, GPR15, which is induced by AHR stimulation via 10-CL-BBQ. This synergistic process happens under physiological settings, as both retinoic acid (vitamin A metabolite) and AHR ligands (produced by various endogenous pathways as well as gut microbiota) are present in the colon-associated lymphoid organs.

By administering compounds that simultaneously modulate RAR signaling and AHR signaling, lymphocyte homing can be increased or decreased to the colon to benefit various disease settings.

RAR agonists include: all-trans retinoic acid (ATRA), AM80, AM580, etc.

AHR agonists include: 10-CL-BBQ, L-kynurenine, ITE, FICZ, indirubin, VAF347, other ligands such as flavonoids, carotinoids, glucobrassin metabolites, tryptophan metabolites, etc.

### Vaccines for Enteric Pathogens

By combining RAR agonists, AHR agonists and vaccines, protective antigen-specific T cells (resident memory T cells) and antibodies can be established in the colon, which hold the potential to increase protective capacity of the vaccines.

### Colon Cancer Treatment

Combined administration of RAR agonists and AHR agonists, either alone or combined with other treatment regimens such as cancer vaccines and checkpoint blockade, can potentially lead to better colon cancer outcomes, through increasing the numbers of activated lymphocytes (CD8+ T cells) in the colon.

### Inflammatory Bowel Disease Treatment

Combined administration of RAR agonists and AHR agonists, together with treatment setting to promote tolerance, holds the potential to improve IBD outcomes by targeting more Tregs to the colon. Conversely, combined administration of RAR inhibitors and AHR inhibitors might block inflammatory lymphocyte homing to the colon.

When given in free form, the aforementioned compounds (RAR agonists, RAR inhibitors, AHR agonists, AHR inhibitors) might have undesirable toxic effects. To address this concern, nanocarrier delivery platforms (such as nanopolymers) can be used. These strategies deliver compounds more effectively to the lymph nodes and promote retention in the lymph nodes, which can help reduce the doses needed. Another strategy is to deliver mRNA formulations that encode key enzymes in the production of RAR and AHR modulating metabolites. For RAR targeting, mRNA encoding retinaldehyde dehydrogenase family enzymes (RALDH1, RALDH2, RALDH3) can be delivered, which produce retinoic acid from retinal. For AHR targeting, mRNA encoding tryptophan pathway enzymes, such as indoleamine 2,3-dioxygenases (IDO1, IDO2) and tryptophan 2,3-dioxygenase (TDO) can be delivered.

### REFERENCES

ⁱ Von Andrian, U. H., & Mackay, C. R. (2000). T-cell function and migration-two sides of the same coin. New England Journal of Medicine, 343(14), 1020-1034.
ⁱⁱ Mora, J. R., Cheng, G., Picarella, D., Briskin, M., Buchanan, N., & von Andrian, U. H. (2005). Reciprocal and dynamic control of CD8 T cell homing by dendritic cells from skin- and gut-associated lymphoid tissues. The Journal of experimental medicine, 201(2), 303-316.
ⁱⁱⁱ Mora, J. R., Bono, M. R., Manjunath, N., Weninger, W., Cavanagh, L. L., Rosemblatt, M., & Von Andrian, U. H. (2003). Selective imprinting of gut-homing T cells by Peyer's patch dendritic cells. Nature, 424(6944), 88-93.
^{iv} Iwata, M., Hirakiyama, A., Eshima, Y., Kagechika, H., Kato, C., & Song, S. Y. (2004). Retinoic acid imprints gut-homing specificity on T cells. Immunity, 21(4), 527-538.
^{v} Svensson, M., Johansson-Lindbom, B., Zapata, F., Jaensson, E., Austenaa, L. M., Blomhoff, R., & Agace, W. W. (2008). Retinoic acid receptor signaling levels and antigen dose regulate gut homing receptor expression on CD8+ T cells. Mucosal immunology, 1(1), 38-48.
^{vi} Dzutsev, A., Hogg, A., Sui, Y., Solaymani-Mohammadi, S., Yu, H., Frey, B., . . . & Berzofsky, J. A. (2017). Differential T cell homing to colon vs. small intestine is imprinted by local CD11c+ APCs that determine homing receptors. Journal of leukocyte biology, 102(6), 1381-1388.
^{vii} Kim, S. V., Xiang, W. V., Kwak, C., Yang, Y., Lin, X. W., Ota, M., . . . & Littman, D. R. (2013). GPR15-mediated homing controls immune homeostasis in the large intestine mucosa. Science, 340(6139), 1456-1459.
^{viii} Littman, D. R., & Kim, S. W. (2016). U.S. Pat. No. 9,376,663. Washington, DC: U.S. Patent and Trademark Office.
^{ix} Kang, S. G., Park, J., Cho, J. Y., Ulrich, B., & Kim, C. H. (2011). Complementary roles of retinoic acid and TGF-β1 in coordinated expression of mucosal integrins by T cells. Mucosal immunology, 4(1), 66-82.
^{x} Xiong, L., Dean, J. W., Fu, Z., Oliff, K. N., Bostick, J. W., Ye, J., . . . & Zhou, L. (2020). Ahr-Foxp3-RORγt axis controls gut homing of CD4+ T cells by regulating GPR15. Science Immunology, 5(48).
^{xi} Ehrlich, A. K., Pennington, J. M., Tilton, S., Wang, X., Marshall, N. B., Rohlman, D., . . . & Kolluri, S. K. (2017). AhR activation increases IL-2 production by alloreactive CD4+ T cells initiating the differentiation of mucosal-homing Tim3+ Lag3+ Tr1 cells. European journal of immunology, 47(11), 1989-2001.

### INCORPORATION BY REFERENCE

The entire disclosure of each of the patent documents, including patent application documents, scientific articles, governmental reports, websites, and other references referred to herein is incorporated by reference herein in its entirety for all purposes. In case of a conflict in terminology, the present specification controls. All sequence listings, or Seq. ID. Numbers, disclosed herein are incorporated herein in their entirety.

The following references, to the extent that they provide exemplary procedural or other details supplementary to those set forth herein, are specifically incorporated herein by reference.

Although illustrative embodiments of the present invention have been described herein, it should be understood that the invention is not limited to those described, and that various other changes or modifications may be made by one skilled in the art without departing from the scope or spirit of the invention.

We claim:

1. A method of increasing the level of CD8+ T cells in the colon and/or small intestine, the method comprising administering to a subject in need thereof a therapeutically effective amount of a retinoic acid receptor (RAR) agonist and/or an aryl hydrocarbon receptor (AHR) agonist,
thereby increasing the level CD8+ T cells in the colon and/or small intestine compared to a control.

2. A method of increasing trafficking of CD8+ T cells to the colon and/or small intestine, the method comprising administering to a subject in need thereof a therapeutically effective amount of a retinoic acid receptor (RAR) agonist and/or an aryl hydrocarbon receptor (AHR) agonist,
thereby increasing trafficking of CD8+ T cells to the colon and/or small intestine compared to a control.

3. The method of claim 1 or 2, further comprising administering to the subject a therapeutic agent.

4. The method of any one of claims 1-3, wherein the level of CD8+ T cells is increased between about 5% to about 70%, about 10% to about 50%, about 20% to about 30%, about 55% to about 65%, or about 15% to about 40% in the colon and/or small intestine compared to a control.

5. The method of any one of the preceding claims, wherein the level of CD8+ T cells is increased at least about 20%, about 25%, about 35%, about 40%, about 45%, about 50%, about 55%, about 60%, or about 65% in the colon and/or small intestine compared to a control.

6. The method of any one of the preceding claims, wherein the control comprises a level of CD8+ T cells in the colon and/or small intestine prior to the administration of RAR agonists and/or AHR agonists.

7. The method of any one of the preceding claims, wherein the CD8+ T cells in the colon and/or small intestine express CD69+CD103+.

8. The method of claim 7, wherein between about 20% to about 100%, about 30% to about 60%, about 40% to about 60%, about 30% to about 70%, about 60% to about 80% of the CD8+ T cells in the colon and/or small intestine express CD69+CD103+.

9. The method of any one of preceding claims, wherein the CD8+ T cells in the colon and/or small intestine express one or more of α4β7, GPR15, and/or CCR9.

10. The method of claim 9, wherein between about 20% to about 100%, about 30% to about 60%, about 40% to about 60%, about 30% to about 70%, about 60% to about 80% of the CD8+ T cells in the colon and/or small intestine express α4β7 compared to a level of CD8+α4β7+ T cells in the colon and/or small intestine prior to the administration of RAR agonists and/or AHR agonists.

11. The method of claim 9 or 10, wherein between about 20% to about 100%, about 30% to about 60%, about 40% to about 60%, about 30% to about 70%, about 60% to about 80% of the CD8+ T cells express GPR15 compared to a level of CD8+GPR15+ T cells in the colon and/or small intestine prior to the administration of RAR agonists and/or AHR agonists.

12. The method of any one of claims 9-11, wherein between about 20% to about 100%, about 30% to about 60%, about 40% to about 60%, about 30% to about 70%, about 60% to about 80% of the CD8+ T cells express CCR9 compared to a level of CD8+CCR9+ T cells in the colon and/or small intestine prior to the administration of RAR agonists and/or AHR agonists.

13. The method of any one of the preceding claims, further wherein the level of circulating memory T cells expressing CXCR3 and CX3CR1 are increased in the blood compared to a level of CXCR3+CX3CR1+ memory T cells in the blood prior to the administration of RAR agonists and/or AHR agonists.

14. A method of increasing the efficacy of a therapeutic agent, the method comprising administering a retinoic acid receptor (RAR) agonist and/or an aryl hydrocarbon receptor (AHR) agonist before, concomitantly, or after administering the therapeutic agent,
thereby increasing the efficacy of the therapeutic agent compared to a control.

15. The method of claim 14, wherein the control comprises a level of efficacy of the therapeutic agent prior to the administration of RAR agonists and/or AHR agonists.

16. A method for treating a disease or disorder of the colon and/or small intestine, the method comprising administering to a subject in need thereof a therapeutically effective amount of a retinoic acid receptor (RAR) agonist and/or an aryl hydrocarbon receptor (AHR) agonist,
thereby treating the disease or disorder of the colon and/or small intestine.

17. The method of claim 16, further comprising administering to the subject a therapeutic agent.

18. The method of claim 16 or 17, wherein the disease or disorder of the colon or small intestine is a cancer, an autoimmune disease, or an enteric pathogenic infection.

19. The method of claim 18, wherein the cancer is colorectal cancer, adenocarcinoma, sarcoma, carcinoid tumor, gastrointestinal stromal tumor (GIST), or intestinal lymphoma.

20. The method of claim 18, wherein the autoimmune disease is Crohn's disease, ulcerative colitis, celiac disease, autoimmune enteropathy, eosinophilic colitis, Behcet's disease, or autoimmune gastritis.

21. The method of claim 18, wherein the enteric pathogenic infection is Salmonella, Shigella, Clostidioides difficile, Campylobacter jejuni, Vibrio cholera, Yersinia enterocolitica, Escherichia coli, Listeria monocytogenes, Rotavirus, Poliovirus, or Norovirus.

22. The method of claim 21, wherein the enteric pathogenic infection is Listeria monocytogenes.

23. The method of any one of claims 3-15 and 17-22, wherein the therapeutic agent is a vaccine, anti-inflammatory, chemotherapeutic, probiotic and/or an antibiotic.

24. The method of any one of the preceding claims, wherein the RAR agonist is selected from the group consisting of all-trans retinoic acid (ATRA), AM80, AM580, AC 261066, Adapalene, BMS 753, BMS 961, CD 1530, CD2314, CD437, ch 55, DC271, retinoic acid, TTNPB, etretinate, tazarotene, tamibarotene, and a combination thereof.

25. The method of any one of the preceding claims, wherein the AHR agonist is selected from the group consisting of 10-CL-BBQ, L-kynurenine, ITE, FICZ, indirubin, VAF347, a flavonoid, a carotinoid, a glucobrassin metabolite, a tryptophan metabolite, and a combination thereof.

26. The method of any one of claims 1-25, comprising administering to the subject the RAR agonist.

27. The method of claim 26, wherein administering the RAR agonist results in an increase in the level of CD8+ T cells in the small intestine.

28. The method of any one of claims 1-25, comprising administering to the subject the AHR agonist.

29. The method of claim 28, wherein administering the AHR agonist results in an increase in the level of CD8+ T cells in the colon.

30. The method of any one of claims 1-25, comprising administering a combination of the RAR agonist and the AHR agonist to the subject.

31. The method of any one of the preceding claims, wherein the RAR agonist and/or the AHR agonist are encapsulated in a nanoparticle comprising one or more polymers.

32. The method of claim 31, wherein the one or more polymers comprise polylactic-co-glycolic acid (PLGA), polyethylene glycol (PEG), poly ε-caprolactone (PCL), poly lactic acid (PLA), chitosan, dextran, acetylated-dextran (AcDex), or a combination thereof.

33. The method of claim 32, wherein the one or more polymers comprise AcDex.

34. The method of any one of claims 1-25, wherein the RAR agonist and/or the AHR agonist, or the RAR agonist and/or the AHR agonist encapsulated in a nanoparticle are formulated for subcutaneous injection.

35. The method of any one of claims 1-13 and 16-35, wherein the RAR agonist and/or the AHR agonist and the therapeutic agent are administered concomitantly.

36. The method of any one of claims 1-13 and 16-35, wherein the RAR agonist and/or the AHR agonist and the therapeutic agent are administered sequentially.

37. The method of claim 36, wherein the RAR agonist and/or the AHR agonist are administered after the therapeutic agent.

38. The method of claim 36, wherein the RAR agonist and/or the AHR agonist are administered before the therapeutic agent.

39. The method of any one of the preceding claims, wherein the RAR agonist and/or the AHR agonist are administered twice a day, once a day, once every two days, once a week, twice a week, once a month, once every two months, or once every six months.

40. The method of claim 39, wherein the RAR agonist and/or the AHR agonist are administered for 1 week to 1 year.

41. The method of claim 30, wherein the RAR agonist and the AHR agonist are administered separately.

42. The method of claim 30, wherein the RAR agonist is administered prior to the AHR agonist.

43. The method of claim 30, wherein the AHR agonist is administered for more doses than the RAR agonist.

44. A method of decreasing the level of CD8+ T cells in the colon and/or small intestine, the method comprising administering to a subject in need thereof a therapeutically effective amount of a retinoic acid receptor (RAR) inhibitor and/or an aryl hydrocarbon receptor (AHR) inhibitor,
thereby decreasing the level CD8+ T cells in the colon and/or small intestine compared to a control.

45. A method of decreasing trafficking of CD8+ T cells to the colon and/or small intestine, the method comprising administering to a subject in need thereof a therapeutically effective amount of a retinoic acid receptor (RAR) inhibitor and/or an aryl hydrocarbon receptor (AHR) inhibitor,
thereby decreasing trafficking of CD8+ T cells to the colon and/or small intestine compared to a control.

46. A method for treating an inflammatory disease or disorder of the colon and/or small intestine, the method comprising administering to a subject in need thereof a therapeutically effective amount of a retinoic acid receptor (RAR) inhibitor and/or an aryl hydrocarbon receptor (AHR) inhibitor,
thereby treating the inflammatory disease or disorder of the colon and/or small intestine.

47. The method of any one of claims 44-46, further comprising administering to the subject a therapeutic agent.

48. The method of any one of claims 44-45, wherein the control comprises a level of CD8+ T cells in the colon and/or small intestine prior to the administration of the RAR inhibitor and/or the AHR inhibitor.

49. The method of any one of claims 46-48, wherein the inflammatory disease or disorder of the colon or small intestine an autoimmune disease.

50. The method of any one of claims 44-49, wherein the RAR inhibitor is selected from the group consisting of 4-[(1E)-2-[5,6-Dihydro-5,5-dimethyl-8-(phenylethynyl)-2-naphthalenyl]ethenyl]-benzoic acid (BMS 493), 4-[2-(5,6,7,8-Tetrahydro-5,5,8,8-tetramethyl-2-naphthalenyl)ethynyl)-benzoic acid (EC 23), AGN 193109-d7,4-[2-[5,6-Dihydro-5,5-dimethyl-8-(4-methylphenyl)-2-naphthalenyl]ethynyl]benzoic Acid Sodium Salt (AGN 193109 Sodium Salt), 4-[[[5,6-Dihydro-5,5-dimethyl-8-(3-quinolinyl)-2-naphthalenyl]carbonyl]amino]benzoic acid (BMS 1695614), 4-[6-[(2-Methoxyethoxy)methoxy]-7-tricyclo[3.3.1.13,7]dec-1-yl-2-naphthalenyl)benzoic acid (CD2665), 4-[5-[8-(1-Methylethyl)-4-phenyl-2-quinolinyl]-1H-pyrrolo-2-benzoic acid (ER50891), 4-(7,8,9,10-Tetrahydro-5,7,7,10,10-pentamethyl-5H-benzo[e]naphtho[2,3-b][1,4]diazepin-13-yl)benzoic acid (LE135), 4-[5-[3,5-Bis(1,1-dimethylethyl)phenyl]-1-[4-[(4-methyl-1-piperazinyl)carbonyl]phenyl]-1H-pyrazol-3-yl]benzoic acid (LY2955303), 6-[2-(5,6,7,8-Tetrahydro-5,5,8,8-tetramethyl-2-naphthalenyl)-1,3-dithiolan-2-yl]-2-naphthalenecarboxylic acid (MM11253), UVI3003, and a combination thereof.

51. The method of any one of claims 44-50, wherein the AHR inhibitor is selected from the group consisting of (S)-6-(4-chlorophenyl)-N-(1-hydroxypropan-2-yl)-2-(1-methyl-1H-pyrazol-4-yl)-3-oxo-2,3-dihydropyridazine-4-carboxamide (BAY 2416964), (R)-N-(2-(5-fluoropyridin-3-yl)-8-isopropylpyrazolo[1,5-a][1,3,5]triazin-4-yl)-2,3,4,9-tetrahydro-1H-carbazol-3-amine (IK-175), N-(2-(1H-indol-3-yl)ethyl)-5-(5-fluoropyridin-3-yl)-3-methylpyrazolo[1,5-a]pyrimidin-7-amine (KYN-101), (1S,2S)-2-(3,4-dichlorobenzoyl)cyclopropane-1-carboxylic acid (UPF-648), brevifolincarboxylic acid, 26-Deoxyactein, hCYP1B1-IN-2, AHR-IN-1,1,2,3,4,7,8,9-Heptachlorodibenzofuran (1,2,3,4,7,8,9-HpCDF), 1,2,3,4,7,8-Hexachlorodibenzofuran (1,2,3,4,7,8-HxCDF), and a combination thereof.
